SEQUENCE LISTING (6 sequences)

SEQ ID NO: 1 - protein, 38 aa, Artificial Sequence
  Synthetic peptide
  MISC_FEATURE (1)..(38): des-Pro38-exendin-4(1-39)
HGEGTFTSDLSKQMEEEAVRLFIEWLKNGGPSSGAPPS

SEQ ID NO: 2 - protein, 38 aa, Artificial Sequence
  Synthetic peptide
  MISC_FEATURE (1)..(38): des-Ser39-exendin-4
HGEGTFTSDLSKQMEEEAVRLFIEWLKNGGPSSGAPPP

SEQ ID NO: 3 - protein, 39 aa, Heloderma suspectum
  MISC_FEATURE (1)..(39): Exendin-4 (1-39)
HGEGTFTSDLSKQMEEEAVRLFIEWLKNGGPSSGAPPPS

SEQ ID NO: 4 - protein, 38 aa, Artificial Sequence
  Synthetic peptide
  misc_feature (1)..(38): des-Pro38 / des-Ser39
  misc_feature (38)..(38): Xaa = Ser or Pro
HGEGTFTSDLSKQMEEEAVRLFIEWLKNGGPSSGAPPX

SEQ ID NO: 5 - protein, 44 aa, Artificial Sequence
  Synthetic peptide
  MISC_FEATURE (1)..(44): Lixisenatide (Lyxumia) des-Pro38-Exendin-4-SIP
  MISC_FEATURE (44)..(44): C-terminal amidated (-NH2)
HGEGTFTSDLSKQMEEEAVRLFIEWLKNGGPSSGAPPSKKKKKK

SEQ ID NO: 6 - protein, 30 aa, Homo sapiens
  MISC_FEATURE (1)..(30): GLP-1 (7-36)
HAEGTFTSDVSSYLEGQAAKEFIAWLVKGR

(19) United States
(12) Patent Application Publication
(10) Pub. No.: US 2023/0303640 A1
(43) Pub. Date: Sep. 28, 2023

(54) EXENDIN-4 PEPTIDE ANALOGS

(71) Applicant: TXP Pharma AG, Baar (CH)

(72) Inventors: Thomas Engelbrecht Nordkild JONASSEN, Holte (DK); Thomas BOESEN, Copenhagen Ø (DK)

(21) Appl. No.: 18/020,813

(22) PCT Filed: Aug. 10, 2021
(86) PCT No.: PCT/EP2021/072251

§ 371 Date: Feb. 10, 2023

(30) Foreign Application Priority Data
Aug. 12, 2020 (DK) PA 2020 70526

(51) Int. Cl. C07K 14/46; A61P 3/10

(52) U.S. Cl. CPC C07K 14/46; A61P 3/10

(57) ABSTRACT

The present invention relates to exendin-4 peptide analogues comprising an exendin-4 peptide and functional variants thereof, and one or more branched amino acid probes.

### TECHNICAL FIELD

The present invention relates to exendin-4 peptide analogues comprising an exendin-4 peptide and one or more branched amino acid probes.

### BACKGROUND

Proteins and peptides are widely employed for therapeutic purposes whether in their native forms, variant forms or analogues thereof. Protein therapeutics tend to be specific for their targets, leading to potentially fewer side effects, but often with lower bioavailability, poorer membrane permeability, and metabolic instability, as compared to small molecules. Protein-based drugs are generally referred to as ‘biologics’ and include molecules such as insulin, growth factors, and engineered antibodies.

Proteinaceous molecules typically require injection; nevertheless, biologics have been an extremely successful class of therapeutics including antibodies for treatment of arthritis and various cancers, soluble proteins for diabetes, myelosuppression and renal anemia; as well as short injectable peptides for multiple sclerosis, cancers, endometriosis and fibroids and acromegaly.

Peptides represent a class of molecules that have the specificity and potency of larger protein biologics, but are smaller in size and more accessible and cheaper to manufacture using chemical methods, thus potentially combining some of the advantages of proteins with those of small molecules.

Protein and peptide compounds can be modified in various ways in order to improve one or more features of the compound, or address one or more potential draw-backs of the compound. For example, a stabilizing peptide sequence may be added to the N- and/or C-terminus of pharmacologically active peptides potentially making them less susceptible to degradation (WO 99/46283). Further, a linear amino acid probe of 6 amino acids selected from Lys or Glu added to the N-terminus of α-MSH potentially increases efficacy compared to the native peptide (WO 07/22774). Known peptide-drug conjugates further include addition of polycationic peptides CPP (cell-penetrating peptides) to improve transport across the cell lipid bi-layer. Analogues of α-MSH and γ-MSH comprising an N-terminally branched amino acid probe are disclosed in WO 2014/060606 and EP 2722340. Peptide analogues with branched amino acid probes are disclosed in WO/2015/162485.

### SUMMARY

The present invention provides exendin-4 peptide analogues comprising one or more branched amino acid probes (abbreviated BAP herein).

The attachment of an amino acid probe, such as a linear Structure Induced Probe (SIP), or a branched amino acid probe, to a biologically active peptide of interest is known to improve or alter an external effect of the active peptide (including for example increased stability, reduced degradation, altered configuration and/or altered solubility), as well as having potential in improving or increasing an inherent effect of the active peptide. For instance, addition of (Lys)₆ to the N-terminus of α-MSH and addition of (Lys)₆ to the C-terminus of des-pro³⁸-exendin-4 (lixisenatide) have been tested with promising results. Adding a branched amino acid probe has also shown promise.

The inventors show herein that the properties of exendin-4 modified by attachment of a branched amino acid probe are improved to achieve increased agonist activity to a surprisingly high extent. As shown herein, des-pro³⁸-exendin-4 modified by attachment of a C-terminal BAP is 10-fold more potent than C-terminal SIP-attachment (lixisenatide), and more potent than BAP-attachment of GLP-1(7-37).

It is an aspect to provide an exendin-4 peptide analogue comprising an exendin-4 peptide and one or more branched amino acid probes,
wherein said branched amino acid probe comprises a first amino alkyl amino acid residue,
said first amino alkyl amino acid residue optionally being covalently linked to a second amino alkyl amino acid residue, or to a second and a third amino alkyl amino acid residue, to form a linear chain of 2 or 3 amino alkyl amino acid residues,
wherein the side chain of one or more of said first, second and/or third amino alkyl amino acid residues are each modified by attaching to the side chain amino group a molecule independently selected from the group consisting of AAA_{q}-AAA; (aa₃)ₚ-AAA_{q}; AAA_{q}-(aa₃)ₚ; [(aa₃)-AAA]ₚ and [AAA-(aa₃)]ₚ;
wherein q is a number selected from 0, 1, 2 and 3; p is a number selected from 1, 2 and 3; AAA is an amino alkyl amino acid residue; and (aa₃) is an amino acid residue independently selected from Arg, His, Gly and Ala,
wherein said first amino alkyl amino acid residue is covalently linked to the N-terminus of said exendin-4 peptide analogue, covalently linked to the C-terminus of said exendin-4 peptide analogue, and/or covalently linked to the side chain amino group of an amino alkyl amino acid residue within said exendin-4 peptide analogue,
with the proviso that said branched amino acid probe consists of 2 to 9 amino acid residues, and
wherein said exendin-4 peptide is selected from the group consisting of des-Pro³⁸-exendin-4(1-39) (SEQ ID NO:1), des-Ser³⁹-exendin-4(1-39) (SEQ ID NO:2) and exendin-4(1-39) (SEQ ID NO:3) or a functional variant thereof.

Also encompassed are pharmaceutical compositions comprising the exendin-4 peptide analogues as disclosed herein, as well as the exendin-4 peptide analogues for use as a medicament.

In one embodiment there is provided an exendin-4 peptide analogue for use in the treatment of diabetes mellitus type 2 or obesity.

### DESCRIPTION OF DRAWINGS

FIG. 1: Schematic representation of the branched amino acid probe Ac-(Ac-LysLys)Lys-, showing the first amino alkyl amino acid residue being a lysine residue (Lys₁), covalently linked to the N-terminus of a peptide sequence via a regular peptide bond, said first lysine being acetylated (COCH₃), and said first lysine modified by attaching to the ε-amino group of said first lysine residue two further lysine residues wherein one is also acetylated (the outermost).

FIG. 2: Receptor efficacy against the human GLP-1 receptor of GLP-1 analogues:

GLP-1 (7-36) having sequence His-Ala-Glu-Gly-Thr-Phe-Thr-Ser-Asp-Val-Ser-Ser-TyrLeu-Glu-Gly-Gln-Ala-Ala-Lys-Glu-Phe-Ile-Ala-Trp-Leu-Val-Lys-Gly-Arg-Gly (SEQ ID NO:6);

Lixisenatide (Lyxumia): des-Pro³⁸-Exendin-4-SIP having the sequence His-Gly-GluGly-Thr-Phe-Thr-Ser-Asp-Leu-Ser-Lys-Gln-Met-Glu-Glu-Glu-Ala-Val-Arg-Leu-Phe-Ile-Glu-Trp-Leu-Lys-Asn-Gly-Gly-Pro-Ser-Ser-Gly-Ala-Pro-Pro-Ser-(Lys)₆-NH₂ (SEQ ID NO:5); and

Analogue 1: des-Pro³⁸-Exendin-4-BAP having sequence His-Gly-Glu-Gly-Thr-Phe-ThrSer-Asp-Leu-Ser-Lys-Gln-Met-Glu-Glu-Glu-Ala-Val-Arg-Leu-Phe-Ile-Glu-Trp-Leu-Lys-Asn-Gly-Gly-Pro-Ser-Ser-Gly-Ala-Pro-Pro-Ser-(Ac-Lys-Lys)Lys-NH₂ (SEQ ID NO:1-(Ac-Lys-Lys)Lys-NH2)

See Example 2 for results and details.

FIG. 3: Receptor efficacy against the human GLP-1 receptor of GLP-1 analogues:

GLP-1 (7-36) having sequence His-Ala-Glu-Gly-Thr-Phe-Thr-Ser-Asp-Val-Ser-Ser-TyrLeu-Glu-Gly-Gln-Ala-Ala-Lys-Glu-Phe-Ile-Ala-Trp-Leu-Val-Lys-Gly-Arg-Gly (SEQ ID NO:6);

Analogue 2: having the sequence Ac-His-Ala-Glu-Gly-Thr-Phe-Thr-Ser-Asp-Val-SerSer-Tyr-Leu-Glu-Gly-Gln-Ala-Ala-Lys-Glu-Phe-Ile-Ala-Trp-Leu-Val-Lys-Gly-Arg-Gly-(Lys-Lys-Ac)Lys-NH₂; and

Analogue 3: having the sequence Ac-His-Gly-Glu-Gly-Thr-Phe-Thr-Ser-Asp-Val-SerSer-Tyr-Leu-Glu-Gly-Gln-Ala-Ala-Lys-Glu-Phe-Ile-Ala-Trp-Leu-Val-Lys-Gly-Arg-Gly(Lys-Lys-Ac)Lys-NH₂.

Data is included and detailed in PCT/IB2015/000553 (WO/2015/162485).

### DETAILED DESCRIPTION

Exendin-4 Peptide Analogues

Exendin-4, originally isolated from Heloderma suspectum venom, is a glucagon-like peptide-1 (GLP-1) receptor agonist, i.e. it interacts with GLP-1 receptor (GLP1R).

Exendin-4 is known to mimic the effects of the incretin hormone GLP-1, which is released from the intestine in response to food intake. Effects include increasing insulin secretion, decreasing glucagon release, increasing satiety, and slowing gastric emptying.

Exendin-4 (1-39) has the sequence His-Gly-Glu-Gly-Thr-Phe-Thr-Ser-Asp-Leu-SerLys-Gln-Met-Glu-Glu-Glu-Ala-Val-Arg-Leu-Phe-Ile-Glu-Trp-Leu-Lys-Asn-Gly-Gly-ProSer-Ser-Gly-Ala-Pro-Pro-Pro-Ser (SEQ ID NO:3). It is processed from a longer peptide of 87 amino acids comprising a signal peptide (amino acids 1-23), a propeptide (amino acids 24-45) and a peptide (amino acids 48-86) corresponding to exendin-4 (1-39) in mature form. Exendin-4 is detailed at UniProtKB—P26349 (EXE4_HELSU).

Exenatide is a synthetic version of exendin-4, which is approved for the treatment of diabetes mellitus type 2. Exenatide (Byetta) is administered twice daily, or once weekly (Bydureon, extended-release exenatide).

Lixisenatide (trade name Lyxumia) is a once-daily injectable GLP-1 receptor agonist for the treatment of diabetes. Lixisenatide (des-Pro³⁸-exendin-4-SIP) is a variant of exendin-4 omitting proline at position 38 and adding six linear lysine residues at the C-terminus. The 6 lysines are known as a Structure Induced Probe or SIP.

Lixisenatide has the sequence H-His-Gly-Glu-Gly-Thr-Phe-Thr-Ser-Asp-Leu-Ser-LysGln-Met-Glu-Glu-Glu-Ala-Val-Arg-Leu-Phe-Ile-Glu-Trp-Leu-Lys-Asn-Gly-Gly-Pro-Ser-Ser-Gly-Ala-Pro-Pro-Ser-(Lys)₆-NH₂ (SEQ ID NO:5).

It is an aspect to provide an exendin-4 peptide modified by addition of one or more branched amino acid probes. Thus in one embodiment the exendin-4 peptide analogues are conjugates comprising an exendin-4 peptide and one or more branched amino acid probes. An exendin-4 peptide refers to fragments and variants of native exendin-4, as specified herein.

In some embodiments, the exendin-4 peptide analogues as provided herein have certain improved properties compared to the corresponding native or unconjugated exendin-4 peptide; and/or compared to otherwise modified exendin-4 peptides. In one embodiment the exendin-4 peptide analogues provided herein have increased binding affinity and/or activation of one or more relevant receptors, such as GLP-1R. In another embodiment, the exendin-4 peptide analogues provided herein are more stable, such as less susceptible to proteases. Still further, in one embodiment the exendin-4 peptide analogues have higher solubility.

It is an aspect to provide an exendin-4 peptide analogue comprising an exendin-4 peptide and one or more branched amino acid probes,
wherein said branched amino acid probe comprises a first amino alkyl amino acid residue,
said first amino alkyl amino acid residue optionally being covalently linked to a second amino alkyl amino acid residue, or to a second and a third amino alkyl amino acid residue, to form a linear chain of 2 or 3 amino alkyl amino acid residues,
wherein the side chain of one or more of said first, second and/or third amino alkyl amino acid residues are each modified by attaching to the side chain amino group a molecule independently selected from the group consisting of AAA_{q}-AAA; (aa₃)ₚ-AAA_{q}; AAA_{q}-(aa₃)ₚ; [(aa₃)-AAA]ₚ and [AAA-(aa₃)]ₚ;
wherein q is a number selected from 0, 1, 2 and 3; p is a number selected from 1, 2 and 3; AAA is an amino alkyl amino acid residue; and (aa₃) is an amino acid residue independently selected from Arg, His, Gly and Ala,
wherein said first amino alkyl amino acid residue is covalently linked to the N-terminus of said exendin-4 peptide, covalently linked to the C-terminus of said exendin-4 peptide, and/or covalently linked to the side chain amino group of an amino alkyl amino acid residue within said exendin-4 peptide,
with the proviso that said branched amino acid probe consists of 2 to 9 amino acid residues; and
wherein said exendin-4 peptide is selected from the group consisting of des-Pro³⁸-exendin-4(1-39) (SEQ ID NO:1), des-Ser³⁹-exendin-4(1-39) (SEQ ID NO:2) and exendin-4(1-39) (SEQ ID NO:3) or a functional variant thereof.

In one embodiment the N-terminal amino acid residue of the molecule is acetylated at the alpha amino group.

In one embodiment said first amino alkyl amino acid residue is linked by a peptide bond (amide) formed by a reaction of the carboxylic acid, or a derivative thereof, of said first amino alkyl amino acid with the alpha amino group of the N-terminal amino acid residue of said exendin-4 peptide; linked by a peptide bond to the C-terminal amino acid residue of said exendin-4 peptide formed by reacting the alpha amino group of said amino alkyl amino acid residue with the carboxylic acid, or derivative thereof, of said C-terminal amino acid residue; and/or linked to an amino alkyl amino acid residue within said exendin-4 peptide by an amide formed by a reaction of the carboxylic acid, or a derivative thereof, of said first amino alkyl amino acid residue with the alkyl amino group of the amino alkyl amino acid residue.

In one embodiment said first amino alkyl amino acid residue is covalently linked to the N-terminal amino acid of said exendin-4 peptide, covalently linked to the C-terminal amino acid of said exendin-4 peptide, and/or covalently linked to the side chain amino group of a Lys or Orn residue within said exendin-4 peptide.

In one embodiment an amino acid residue being covalently linked to further amino acid residues and/or a peptide in one embodiment means that a peptide bond is present. In another embodiment an amino acid residue being covalently linked to the side chain amino group of an amino alkyl amino acid residue within said exendin-4 peptide means that an amide bond is present.

A peptide bond (amide bond) is a covalent chemical bond formed between two molecules when the carboxyl group of one molecule reacts with the amino group of the other molecule, causing the release of a molecule of H₂O. The process usually occurs between amino acids.

If the branched amino acid probe is to be covalently linked to the N-terminus of said exendin-4 peptide, the N-terminal amino alkyl amino acid residue of the backbone of the branched amino acid probe is preferably acetylated.

If the branched amino acid probe is to be covalently linked to the side chain amino group of an amino alkyl amino acid residue within said exendin-4 peptide, the N-terminal amino alkyl amino acid residue of the backbone of the branched amino acid probe is preferably acetylated.

If the branched amino acid probe is to be covalently linked to the C-terminus of said exendin-4 peptide, the C-terminal amino alkyl amino acid residue of the backbone of the branched amino acid probe is preferably a carboxylic acid, an aldehyde, an ester, or an amide, such as a primary amide; most preferably amidated.

The amino alkyl amino acid residues (or AAA) and the amino acid residues (aa₃) may each be the same (identical) or different (non-identical).

Branched Amino Acid Probe

Amino Alkyl Amino Acid Residue

As defined herein an ‘amino alkyl amino acid residue’ (or AAA) is an amino acid having the conventional amine (—NH₂) and carboxylic acid (—COOH) functional groups, and a side chain covalently linked to the first (alpha-) carbon atom, wherein the side-chain comprises an amino alkyl group (—CₙH₂ₙNH₂).

Thus an amino alkyl amino acid residue (or AAA) is an amino acid with a side chain comprising or consisting of an amino alkyl group (—CₙH₂ₙNH₂), in one embodiment denoted a side chain amino alkyl group.

In one embodiment the side chain alkyl group is derived from the group consisting of methyl (CH₃—), ethyl (C₂H₅—), propyl (C₃H₇—), butyl (C₄H₉—), pentyl (C₅H₁₁—), hexyl (C₆H₁₃—), heptyl (C₇H₁₅—), octyl (C₈H₁₇—), nonyl (C₉H₁₉—), decyl (C₁₀H₂₁—), undecyl (C₁₁H₂₃—) and dodecyl (C₁₂H₂₅—). When an alkyl residue having a specific number of carbons is named, all geometric isomers having that number of carbons are intended to be encompassed; thus, for example, “butyl” is meant to include n-butyl, sec-butyl, isobutyl and t-butyl.

In one embodiment the side chain amino group (NH₂) of said amino alkyl amino acid residue is the amine of methylamine, the amine of ethylamine, the amine of propylamine, the amine of n-butylamine, the amine of pentylamine, the amine of n-hexylamine, the amine of heptylamine, the amine of octylamine, the amine of nonylamine, the amine of decylamine, the amine of undecylamine or the amine of dodecylamine.

In one embodiment the side chain amino alkyl group is selected from the group consisting of methylamine (—CH₂NH₂), ethylamine (—C₂H₄NH₂), propylamine (—C₃H₆NH₂), n-butylamine (—C₄H₈NH₂), pentylamine (—C₅H₁₀NH₂), n-hexylamine (—C₆H₁₂NH₂), heptylamine (—C₇H₁₄NH₂), octylamine (—C₈H₁₆NH₂), nonylamine (—C₉H₁₈NH₂), decylamine (—C₁₀H₂₀NH₂), undecylamine (—C₁₁H₂₂NH₂) and dodecylamine (—C₁₂H₂₄NH₂).

In one embodiment the side chain amino group (NH₂) of said first, second and/or third amino alkyl amino acid residues are each modified by attaching a molecule thereto.

In one embodiment the side chain amino group of said amino alkyl amino acid residue is selected from the group consisting of
the β (beta) amino group (1 methylene in the side chain; methylamine);
the γ (gamma) amino group (2 methylenes in the side chain, ethylamine);
the δ (delta) amino group (3 methylenes in the side chain, propylamine); =ornithine
the ε (epsilon) amino group (4 methylenes in the side chain; n-butylamine); =lysine
the ζ (zeta) amino group (5 methylenes in the side chain; pentylamine);
the η (eta) amino group (6 methylenes in the side chain; n-hexylamine);
the θ (theta) amino group (7 methylenes in the side chain; heptylamine);
the ι (iota) amino group (8 methylenes in the side chain; octylamine);
the κ (kappa) amino group (9 methylenes in the side chain; nonylamine);
the λ (lambda) amino group (10 methylenes in the side chain; decylamine);
the μ (mu) amino group (11 methylenes in the side chain; undecylamine); and
the v (nu) amino group (12 methylenes in the side chain; dodecylamine).

For example, the ε-amino group is covalently linked to the fifth carbon beginning from (including) the α-carbon, which α-carbon is covalently linked to the carboxyl (C═OOH) group.

An amino alkyl amino acid residue wherein the side chain is n-butylamine and the side chain amino group is the ε (epsilon) amino group is lysine (Lys, K).

Likewise, the δ-amino group is covalently linked to the fourth carbon beginning from the α-carbon.

An amino alkyl amino acid residue wherein the side chain is propylamine and the side chain amino group is the δ (delta) amino group is ornithine (Orn).

Ornithine is formed in cells by deguanidation of arginine. While it is not used in proteinogenesis in vivo it is a participant in several enzyme pathways and appears to play a role in nitrogen balance in vivo as it can be gaunidated enzymatically to form arginine.

Any amino acid as defined herein may be in the L- or D-configuration. If nothing is specified, reference to the L-isomeric form is preferably meant.

It follows that the amino alkyl amino acid residues as disclosed herein in one embodiment are individually in the L- or D-configuration. In one embodiment the amino alkyl amino acid residues are in the L-configuration.

In one embodiment the amino alkyl amino acid residues comprised in the branched amino acid probe are individually selected from the group consisting of lysine and ornithine.

In one embodiment the amino alkyl amino acid residues are selected from the group consisting of lysine and D-lysine. In a particular embodiment the amino alkyl amino acid residues of the invention are lysine residues.

In one embodiment the amino alkyl amino acid residues are selected from the group consisting ornithine and D-ornithine.

In one embodiment there is provided an exendin-4 peptide analogue comprising an exendin-4 peptide and one or more branched amino acid probes,
wherein said branched amino acid probe comprises a first amino acid residue selected from lysine and ornithine,
said first amino acid residue optionally being covalently linked to a second, or to a second and a third amino acid residue selected from lysine or ornithine, to form a linear chain of 2 or 3 lysine or ornithine residues,
wherein the side chain of one or more of said first, second and/or third lysine or ornithine residues are modified by attaching to the δ-amino group (ornithine) or the ε-amino group (lysine) a molecule independently selected from the group consisting of
Lys_{q}-Lys; (aa₃)ₚ-Lys_{q}; Lys_{q}-(aa₃)ₚ; [(aa₃)-Lys]ₚ; [Lys-(aa₃)]ₚ;
Orn_{q}-Orn; (aa₃)ₚ-Orn_{q}; Orn_{q}-(aa₃)ₚ; [(aa₃)-Orn]ₚ and [Orn-(aa₃)]ₚ;
Ornₚ-Lysₚ; Lysₚ-Ornₚ; [Orn-Lys]ₚ and [Lys-Orn]ₚ;
wherein q is a number selected from 0, 1, 2 and 3; p is a number selected from 1, 2 and 3; and (aa₃) is an amino acid residue independently selected from Arg, His, Gly and Ala,
wherein said first lysine or ornithine residue is covalently linked to the N-terminus of said exendin-4 peptide, covalently linked to the C-terminus of said exendin-4 peptide, and/or covalently linked to the ε-amino group of a lysine residue or the δ-amino group of an ornithine residue within said exendin-4 peptide,
with the proviso that said branched amino acid probe consists of 2 to 9 amino acid residues.

In one embodiment there is provided an exendin-4 peptide analogue comprising an exendin-4 peptide and one or more branched amino acid probes,
wherein said branched amino acid probe comprises a first lysine residue,
said first lysine residue optionally being covalently linked to a second, or to a second and a third lysine residue, to form a linear chain of 2 or 3 lysine residues,
wherein the side chain of one or more of said first, second and/or third lysine residues are modified by attaching to the ε-amino group of said lysine a molecule independently selected from the group consisting of Lys_{q}-Lys; (aa₃)ₚ-Lys_{q}; Lys_{q}-(aa₃)ₚ; [(aa₃)-Lys]ₚ; [Lys-(aa₃)]ₚ; wherein q is a number selected from 0, 1, 2 and 3; p is a number selected from 1, 2 and 3; and (aa₃) is an amino acid residue independently selected from Arg, His, Gly and Ala,
wherein said first lysine residue is covalently linked to the N-terminus of said exendin-4 peptide, covalently linked to the C-terminus of said exendin-4 peptide, and/or covalently linked to the ε-amino group of a lysine or δ-amino group of an ornithine residue within said exendin-4 peptide,
with the proviso that said branched amino acid probe consists of 2 to 9 amino acid residues.

Branching the Probe

A branched amino acid probe as defined herein in one embodiment consists of 2 to 9 amino acid residues.

In one embodiment each of said one or more branched amino acid probe consist of from 2 to 3 amino acid residues, such as from 3 to 4 amino acid residues, for example from 4 to 5 amino acid residues, such as from 5 to 6 amino acid residues, for example from 6 to 7 amino acid residues, such as from 7 to 8 amino acid residues, for example from 8 to 9 amino acid residues.

In one embodiment each of said one or more branched amino acid probe consist of 2 amino acid residues, such as 3 amino acid residues, for example 4 amino acid residues, such as 5 amino acid residues, for example 6 amino acid residues, such as 7 amino acid residues, for example 8 amino acid residues, such as 9 amino acid residues. In a particular embodiment each of said one or more branched amino acid probes consists of 3 amino acid residues.

In one embodiment the branched amino acid probe comprises a first amino alkyl amino acid residue (also denoted AAA₁), which first amino alkyl amino acid residue is connected to an exendin-4 peptide to provide an exendin-4 peptide analogue as defined herein.

In one embodiment the first amino alkyl amino acid of (each of) the one or more branched amino acid probe(s) is covalently linked to the N-terminus of said exendin-4 peptide, covalently linked to the C-terminus of said exendin-4 peptide, and/or covalently linked to the side chain amino group of an amino alkyl amino acid residue within said exendin-4 peptide.

In one embodiment the first amino alkyl amino acid residue of (each of) the one or more branched amino acid probe(s) is covalently linked to the N-terminal amino acid of said exendin-4 peptide, covalently linked to the C-terminal amino acid of said exendin-4 peptide, and/or covalently linked to the side chain of a Lys or Orn residue within said exendin-4 peptide.

In one embodiment the branched amino acid probe comprises a first amino alkyl amino acid residue. In one embodiment the side chain of said first amino alkyl amino acid residue is modified by attaching to the side chain amino group a molecule as defined herein.

In one embodiment the first amino alkyl amino acid of the branched amino acid probe is acetylated at the alpha amino group. In one embodiment the N-terminus of the first amino alkyl amino acid residue of the branched amino acid probe is acetylated.

In one embodiment the N-terminus of the first amino alkyl amino acid residue of the branched amino acid probe is acetylated when the branched amino acid probe comprising said first amino alkyl amino acid residue is covalently linked to the N-terminus of the exendin-4 peptide; or when the branched amino acid probe comprising said first amino alkyl amino acid residue is covalently linked to the side chain amino group of an amino alkyl amino acid residue within said exendin-4 peptide.

In one embodiment the C-terminus of the first amino alkyl amino acid residue of the branched amino acid probe is a carboxylic acid, an aldehyde, an ester, or an amide, such as a primary amide (CONH₂). In a preferred embodiment the C-terminus of the first amino alkyl amino acid residue is amidated.

In one embodiment the C-terminus of the first amino alkyl amino acid residue of the branched amino acid probe is an amide when the branched amino acid probe comprising said first amino alkyl amino acid residue is covalently linked to the C-terminus of the exendin-4 peptide.

In one embodiment said first amino alkyl amino acid residue is covalently linked to a second amino alkyl amino acid residue to form a linear chain of 2 amino alkyl amino acid residues. In one embodiment the alpha-amino group of the second amino alkyl amino acid residue of the branched amino acid probe is acetylated. In one embodiment the N-terminus of the second amino alkyl amino acid residue of the branched amino acid probe is acetylated.

In one embodiment the C-terminus of the second amino alkyl amino acid residue of the branched amino acid probe is a carboxylic acid, an aldehyde, an ester, or an amide, such as a primary amide (CONH₂). In a preferred embodiment the C-terminus of the second amino alkyl amino acid residue is amidated.

In one embodiment said first amino alkyl amino acid residue is covalently linked to a second and (covalently linked to) a third amino alkyl amino acid residue to form a linear chain of 3 amino alkyl amino acid residues.

In one embodiment the alpha-amino group of the third amino alkyl amino acid residue of the branched amino acid probe is acetylated. In one embodiment the N-terminus of the third amino alkyl amino acid residue of the branched amino acid probe is acetylated.

In one embodiment the C-terminal of the third amino alkyl amino acid residue of the branched amino acid probe is a carboxylic acid, an aldehyde, an ester, or an amide, such as a primary amide (CONH₂). In a preferred embodiment the C-terminus of the third amino alkyl amino acid residue is amidated.

In one embodiment the first amino alkyl amino acid residue have both the second and third amino alkyl amino acid residues attached at its amine group. In one embodiment the first amino alkyl amino acid residue have both the second and third amino alkyl amino acid residues covalently linked to its carboxylic acid group. In one embodiment the first amino alkyl amino acid residue have the second amino alkyl amino acid residue attached at its amine group and the third amino alkyl amino acid residue attached at its carboxylic acid group.

The second and third amino alkyl amino acid residues may be denoted AAA₂ and AAA₃, respectively.

In one embodiment each of said first, second and/or third amino alkyl amino acid residues is an amino acid having a side chain amino alkyl group selected from the group consisting of methylamine (—CH₂NH₂), ethylamine (—C₂H₄NH₂), propylamine (—C₃H₆NH₂), n-butylamine (—C₄H₈NH₂), pentylamine (—C₅H₁₀NH₂), n-hexylamine (—C₆H₁₂NH₂), heptylamine (—C₇H₁₄NH₂), octylamine (—C₈H₁₆NH₂), nonylamine (—C₉H₁₈NH₂), decylamine (—C₁₀H₂₀NH₂), undecylamine (—C₁₁H₂₂NH₂) and dodecylamine (—C₁₂H₂₄NH₂).

In one embodiment each of the first, second and/or third amino alkyl amino acid residues of the branched amino acid probe are individually selected from the group consisting of lysine, D-lysine, ornithine and D-ornithine.

In one embodiment each of the first, second and third amino alkyl amino acid residues of the branched amino acid probe are lysine residues (including L-lysine and D-lysine).

In one embodiment the first, the second or the third amino alkyl amino acid residues of the branched amino acid probe are acetylated at the alpha amino group (Ac-AAA) (COCH₃).

In one embodiment, the first, the first and second, and the first, second and third amino alkyl amino acid residues of the branched amino acid probe are referred to as the amino alkyl amino acid backbone of the branched amino acid probe (AAA₁, AAA₁₋₂, AAA₁₋₃).

In one embodiment the first, the first and second, and the first and second and third amino alkyl amino acid residues are each lysine residues. In one embodiment the first, the first and second, and the first, second and third lysine residues of the branched amino acid probe are referred to as the lysine backbone of the branched amino acid probe (Lys₁, Lys₁₋₂, Lys₁₋₃).

In one embodiment the first lysine residue, or the second lysine residue, or the third lysine residue of the lysine backbone of the branched amino acid probe is acetylated at the alpha-amino group (Ac-Lys).

In one embodiment the side chain of one of said first, second and/or third amino alkyl amino acid residues are modified by attaching to the side chain amino group a molecule as defined herein.

In one embodiment the branched amino acid probe comprises a first amino alkyl amino acid residue, wherein the side chain of said first amino alkyl amino acid residue is modified by attaching to the side chain amino group a molecule as defined herein.

In one embodiment the branched amino acid probe comprises a first and a second amino alkyl amino acid residue, wherein the side chain of said first amino alkyl amino acid residue is modified by attaching to the side chain amino group a molecule as defined herein.

In one embodiment the branched amino acid probe comprises a first and a second amino alkyl amino acid residue, wherein the side chain of said second amino alkyl amino acid residue is modified by attaching to the side chain amino group a molecule as defined herein.

In one embodiment the branched amino acid probe comprises a first and a second amino alkyl amino acid residue, wherein the side chains of said first and second amino alkyl amino acid residue are modified by attaching to the side chain amino group a molecule as defined herein.

In one embodiment the side chain of two of said first, second and/or third amino alkyl amino acid residues are modified by attaching to the side chain amino group a molecule as defined herein.

In one embodiment the side chain of all three of the first, second and third amino alkyl amino acid residues are modified by attaching to the side chain amino group a molecule as defined herein.

In one embodiment the side chain of i) the first amino alkyl amino acid residue, ii) the second amino alkyl amino acid residue, iii) the third amino alkyl amino acid residue, iv) the first and the second amino alkyl amino acid residues, v) the first and the third amino alkyl amino acid residues, vi) the second and the third amino alkyl amino acid residues, or vii) the first, the second and the third amino alkyl amino acid residues, are modified by attaching to the side chain amino group a molecule as defined herein.

In one embodiment the first lysine residue, or the second lysine residue, or the third lysine residue, or the first and the second lysine residues, or the first and the third lysine residues, or the second and the third lysine residues, or the first, the second and the third lysine residues of the lysine backbone of the branched amino acid are modified by attaching a molecule to the ε-amino group.

In one embodiment the side chain of one or more of each of said first, second and/or third amino alkyl amino acid residues is modified by attaching to the side chain amino group a molecule independently selected from the group consisting of AAA_{q}-AAA; (aa₃)ₚ-AAA_{q}; AAA_{q}-(aa₃)ₚ; [(aa₃)-AAA]ₚ and [AAA-(aa₃)]ₚ; wherein q is a number selected from 0, 1, 2 and 3; p is a number selected from 1, 2 and 3; AAA is an amino alkyl amino acid residue; and (aa₃) is an amino acid residue independently selected from Arg, His, Gly and Ala. In one embodiment the N-terminal AAA or (aa)₃ of the molecule is acetylated at the alpha amino group.

In one embodiment the side chain of one or more of each of said first, second and/or third amino alkyl amino acid residues is modified by attaching to the side chain amino group a molecule independently selected from the group consisting of
Lys_{q}-Lys; (aa₃)ₚ-Lys_{q}; Lys_{q}-(aa₃)ₚ; [(aa₃)-Lys]ₚ; [Lys-(aa₃)]ₚ;
Orn_{q}-Orn; (aa₃)ₚ-Orn_{q}; Orn_{q}-(aa₃)ₚ; [(aa₃)-Orn]ₚ and [Orn-(aa₃)]ₚ;
Ornₚ-Lysₚ; Lysₚ-Ornₚ; [Orn-Lys]ₚ and [Lys-Orn]ₚ;
wherein q is a number selected from 0, 1, 2 and 3; p is a number selected from 1, 2 and 3; and (aa₃) is an amino acid residue independently selected from Arg, His, Gly and Ala. In one embodiment the N-terminal Lys, Orn or (aa)₃ of the molecule is acetylated at the alpha amino group.

In one embodiment the side chain of one or more of each of said first, second and/or third amino alkyl amino acid residues is modified by attaching to the side chain amino group a molecule independently selected from the group consisting of Lys_{q}-Lys; (aa₃)ₚ-Lys_{q}; Lys_{q}-(aa₃)ₚ; [(aa₃)-Lys]ₚ and [Lys-(aa₃)]ₚ; wherein q is a number selected from 0, 1, 2 and 3; p is a number selected from 1, 2 and 3; Lys is a lysine residue selected from L-Lys and D-Lys; and (aa₃) is an amino acid residue independently selected from Arg, His, Gly and Ala. In one embodiment the N-terminal Lys or (aa)₃ of the molecule is acetylated at the alpha amino group.

In one embodiment the side chain of one or more of each of said first, second and/or third lysine residues of the lysine backbone is modified by attaching to the ε-amino group of the side chain a molecule independently selected from the group consisting of Lys_{q}-Lys; (aa₃)ₚ-Lys_{q}; Lys_{q}-(aa₃)ₚ; [(aa₃)-Lys]ₚ and [Lys-(aa₃)]ₚ; wherein q is a number selected from 0, 1, 2 and 3; p is a number selected from 1, 2 and 3; Lys is a lysine residue selected from L-Lys and D-Lys; and (aa₃) is an amino acid residue independently selected from Arg, His, Gly and Ala. In one embodiment the N-terminal Lys or (aa)₃ of the molecule is acetylated at the alpha amino group.

In one embodiment the side chain of one or more of each of said first, second and/or third lysine residues of the lysine backbone are modified by attaching to the ε-amino group of the side chain a molecule being Lys_{q}-Lys; wherein q is a number selected from 0, 1, 2 and 3. In one embodiment the N-terminal Lys of the molecule is acetylated at the alpha amino group.

In one embodiment, the molecule to be covalently linked to the ε-amino group of the one or more lysine residues of the lysine backbone of the branched amino acid probe are independently selected from the group consisting of Lys_{q}-Lys; (aa₃)ₚ-Lys_{q}; Lys_{q}(aa₃)ₚ; [(aa₃)-Lys]ₚ and [Lys-(aa₃)]ₚ, wherein q is a number selected from 0, 1, 2 and 3; p is a number selected from 1, 2 and 3, and (aa₃) is an amino acid residue independently selected from Arg, His, Gly and Ala. In one embodiment the N-terminal Lys or (aa)₃ of the molecule is acetylated at the alpha amino group.

It follows that in one embodiment the first lysine residue, or the second lysine residue, or the third lysine residue, or the first and the second lysine residues, or the first and the third lysine residues, or the second and the third lysine residues, or the first, the second and the third lysine residues of the branched amino acid probe are each modified by attaching to the ε-amino group(s) a molecule independently selected from the group consisting of Lys_{q}-Lys; (aa₃)ₚ-Lys_{q}; Lys_{q}-(aa₃)ₚ; [(aa₃)-Lys]ₚ and [Lys-(aa₃)]ₚ, wherein q is a number selected from 0, 1, 2 and 3; p is a number selected from 1, 2 and 3, and (aa₃) is an amino acid residue independently selected from Arg, His, Gly and Ala. In one embodiment the N-terminal Lys or (aa)₃ of the molecule is acetylated at the alpha amino group.

In a particular embodiment (aa₃) is an amino acid residue independently selected from Gly and Ala. In a further embodiment, (aa₃) is Gly.

In one embodiment, the molecules to be covalently linked to the side chain amino group(s) of said first, second and/or third alkyl amine amino acid residue are acetylated at the alpha amino group of the N-terminal amino acid residue.

In one embodiment the molecules are independently selected from the group consisting of Ac-AAA_{q}-AAA; Ac-(aa₃)ₚ-AAA_{q}; Ac-AAA_{q}-(aa₃)ₚ; Ac-[(aa₃)-AAA]ₚ and Ac-[AAA-(aa₃)]ₚ; and/or AAA_{q}-AAA; (aa₃)ₚ-AAA_{q}; AAA_{q}-(aa₃)ₚ; [(aa₃)-AAA]ₚ and [AAA-(aa₃)]ₚ

In one embodiment the molecules are independently selected from the group consisting of Ac-Orn_{q}-Orn; Ac-(aa₃)ₚ-Orn_{q}; Ac-Orn_{q}-(aa₃)ₚ; Ac-[(aa₃)-Orn]ₚ; Ac-[Orn-(aa₃)]ₚ; Ac-Ornₚ-Lysₚ; Ac-Lysₚ-Ornₚ; Ac-[Orn-Lys]ₚ and Ac-[Lys-Orn]ₚ; and/or Orn_{q}-Orn; (aa₃)ₚ-Orn_{q}; Orn_{q}-(aa₃)ₚ; [(aa₃)-Orn]ₚ and [Orn-(aa₃)]ₚ; Ornₚ-Lysₚ; Lysₚ-Ornₚ; [Orn-Lys]ₚ and [Lys-Orn]ₚ.

It follows that the molecules are in one embodiment independently selected from the group consisting of Ac-Lys_{q}-Lys; Ac-(aa₃)ₚ-Lys_{q}; Ac-Lys_{q}-(aa₃)ₚ; Ac-[(aa₃)-Lys]ₚ and Ac-[Lys-(aa₃)]ₚ; and/or Lys_{q}-Lys; (aa₃)ₚ-Lys_{q}; Lys_{q}-(aa₃)ₚ; [(aa₃)-Lys]ₚ and [Lys-(aa₃)]ₚ.

In a particular embodiment, the molecule to be covalently linked to the side chain amino group(s) is Ac-AAA_{q}-AAA or AAA_{q}-AAA, wherein q is a number selected from 0, 1, 2 and 3.

It follows that in one embodiment the branched amino acid probe consists of 2 to 9 amino alkyl amino acid residues. In one embodiment said 2 to 9 amino alkyl amino acid residues are individually selected from the group consisting of lysine, D-lysine, ornithine and D-ornithine.

In a particular embodiment, the molecule to be covalently linked to the side chain amino group(s) is Ac-Lys_{q}-Lys or Lys_{q}-Lys, wherein q is a number selected from 0, 1, 2 and 3.

It follows that in one embodiment the branched amino acid probe consists of 2 to 9 lysine residues.

In one embodiment, the branched amino acid probe comprises a maximum of 1, 2, 3 or 4 amino acids selected from Arg, His, Gly and Ala (aa₃), wherein the remaining amino acids are amino alkyl amino acid residues. In another embodiment, the branched amino acid probe comprises a maximum of 1 Arg residue, and/or comprises a maximum of 1 His residue, and/or comprises a maximum of 1 Gly residue, and/or comprises a maximum of 1 Ala residue.

In one embodiment, the molecule to be covalently linked to the side chain amino group(s) of one or more of the first, second and/or third amino alkyl amino acid residues is selected from the group consisting of AAA, Ac-AAA, AAA-AAA, Ac-AAA-AAA, AAA-AAA-AAA, Ac-AAA-AAA-AAA, AAA-AAA-AAA-AAA, Ac-AAA-AAA-AAA-AAA, AAA-Gly-AAA, Ac-AAA-Gly-AAA, AAA-AAA-Gly, Ac-AAA-AAA-Gly, AAA-Gly, Ac-AAA-Gly, AAA-Ala-AAA, Ac-AAA-Ala-AAA, AAA-AAA-Ala, Ac-AAA-AAA-Ala, AAA-Ala, Ac-AAA-Ala, AAA-His-AAA, Ac-AAA-His-AAA, AAA-AAA-His, Ac-AAA-AAA-His, AAA-His, Ac-AAA-His, AAA-Arg-AAA, Ac-AAA-Arg-AAA, AAA-AAA-Arg, Ac-AAA-AAA-Arg, AAA-Arg and Ac-AAA-Arg; wherein AAA is an amino alkyl amino acid residue as specified herein. The above-mentioned AAA, Gly, Ala, His and Arg amino acid residues may each be in the L- or D-conformation.

In one embodiment, the molecule to be covalently linked to the side chain amino group(s) of one or more of the first, second and/or third amino alkyl amino acid residues is selected from the group consisting of Lys, Ac-Lys, Lys-Lys, Ac-Lys-Lys, LysLys-Lys, Ac-Lys-Lys-Lys, Lys-Lys-Lys-Lys, Ac-Lys-Lys-Lys-Lys, Lys-Gly-Lys, Ac-Lys-Gly-Lys, Lys-Lys-Gly, Ac-Lys-Lys-Gly, Lys-Gly, Ac-Lys-Gly, Lys-Ala-Lys, Ac-Lys-Ala-Lys, Lys-Lys-Ala, Ac-Lys-Lys-Ala, Lys-Ala, Ac-Lys-Ala, Lys-His-Lys, Ac-Lys-His-Lys, Lys-Lys-His, Ac-Lys-Lys-His, Lys-His, Ac-Lys-His, Lys-Arg-Lys, Ac-Lys-Arg-Lys, LysLys-Arg, Ac-Lys-Lys-Arg, Lys-Arg and Ac-Lys-Arg.

In a particular embodiment, the molecule to be covalently linked to the ε-amino group(s) of one or more of the first, second and/or third lysine residues is selected from the group consisting of Lys, Ac-Lys, Lys-Lys, Ac-Lys-Lys, Lys-Lys-Lys, Ac-Lys-Lys-Lys, Lys-Lys-Lys-Lys, Ac-Lys-Lys-Lys-Lys, Lys-Gly-Lys, Ac-Lys-Gly-Lys, Lys-Lys-Gly, Ac-Lys-Lys-Gly, Lys-Gly, Ac-Lys-Gly, Lys-Ala-Lys, Ac-Lys-Ala-Lys, Lys-Lys-Ala, Ac-Lys-Lys-Ala, Lys-Ala, Ac-Lys-Ala, Lys-His-Lys, Ac-Lys-His-Lys, Lys-Lys-His, Ac-Lys-Lys-His, Lys-His, Ac-Lys-His, Lys-Arg-Lys, Ac-Lys-Arg-Lys, Lys-Lys-Arg, Ac-Lys-Lys-Arg, Lys-Arg and Ac-Lys-Arg.

In a particular embodiment, the branched amino acid probe comprise or consist of a first lysine residue selected from Lys and D-Lys, said first lysine residue being optionally N-terminally acetylated or C-terminally amidated, wherein said first lysine residue is modified by attaching to the ε-amino group of said first lysine residue a molecule selected from the group consisting of Lys, Ac-Lys, Lys-Lys, Ac-Lys-Lys, Lys-Lys-Lys, Ac-Lys-Lys-Lys, Lys-Lys-Lys-Lys, Ac-Lys-Lys-Lys-Lys, Lys-Gly-Lys, Ac-Lys-Gly-Lys, Lys-Lys-Gly, Ac-Lys-Lys-Gly, Lys-Gly, Ac-Lys-Gly, Lys-Ala-Lys, Ac-Lys-Ala-Lys, Lys-Lys-Ala, Ac-Lys-Lys-Ala, Lys-Ala, Ac-Lys-Ala, Lys-His-Lys, Ac-Lys-His-Lys, Lys-Lys-His, Ac-Lys-Lys-His, Lys-His, Ac-Lys-His, Lys-Arg-Lys, Ac-Lys-Arg-Lys, Lys-Lys-Arg, Ac-Lys-Lys-Arg, Lys-Arg and Ac-Lys-Arg.

In a particular embodiment, the branched amino acid probe comprise or consist of a first and a second lysine residue each selected from Lys and D-Lys, said second lysine residue being optionally N-terminally acetylated or C-terminally amidated, wherein i) said first lysine residue, ii) said second lysine residue, or iii) said first and second residue are each modified by attaching to the ε-amino group of said lysine residue a molecule selected from the group consisting of Lys, Ac-Lys, Lys-Lys, Ac-Lys-Lys, Lys-Lys-Lys, Ac-Lys-Lys-Lys, Lys-Lys-Lys-Lys, Ac-Lys-Lys-Lys-Lys, Lys-Gly-Lys, Ac-Lys-Gly-Lys, Lys-Lys-Gly, Ac-Lys-Lys-Gly, Lys-Gly, Ac-Lys-Gly, Lys-Ala-Lys, Ac-Lys-Ala-Lys, Lys-Lys-Ala, Ac-Lys-Lys-Ala, Lys-Ala, Ac-Lys-Ala, Lys-His-Lys, Ac-Lys-His-Lys, LysLys-His, Ac-Lys-Lys-His, Lys-His, Ac-Lys-His, Lys-Arg-Lys, Ac-Lys-Arg-Lys, Lys-Lys-Arg, Ac-Lys-Lys-Arg, Lys-Arg and Ac-Lys-Arg.

In a particular embodiment, the branched amino acid probe comprise or consist of a first, a second and a third lysine residue each selected from Lys and D-Lys, said third lysine residue being optionally N-terminally acetylated or C-terminally amidated, wherein i) said first lysine residue, ii) said second lysine residue, iii) said third lysine residue, iv) said first and second lysine residue, v) said first and third lysine residue, vi) said second and third lysine residue, or vii) said first, second and third lysine residues are each modified by attaching to the ε-amino group of said lysine residue a molecule selected from the group consisting of Lys, Ac-Lys, Lys-Lys, Ac-Lys-Lys, Lys-Lys-Lys, Ac-Lys-Lys-Lys, Lys-Lys-Lys-Lys, Ac-Lys-Lys-Lys-Lys, Lys-Gly-Lys, Ac-Lys-Gly-Lys, Lys-Lys-Gly, Ac-Lys-Lys-Gly, Lys-Gly, Ac-Lys-Gly, Lys-Ala-Lys, Ac-Lys-Ala-Lys, LysLys-Ala, Ac-Lys-Lys-Ala, Lys-Ala, Ac-Lys-Ala, Lys-His-Lys, Ac-Lys-His-Lys, Lys-Lys-His, Ac-Lys-Lys-His, Lys-His, Ac-Lys-His, Lys-Arg-Lys, Ac-Lys-Arg-Lys, Lys-Lys-Arg, Ac-Lys-Lys-Arg, Lys-Arg and Ac-Lys-Arg.

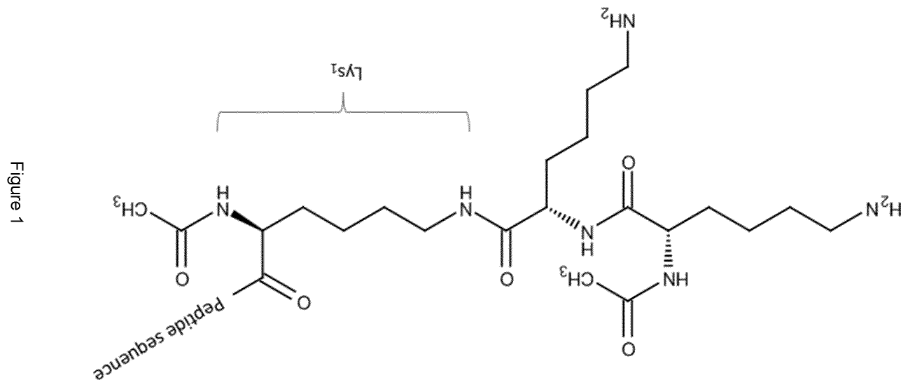

In one embodiment the branched amino acid probe comprises or consists of the formula: Ac-(Ac-Lys-Lys)Lys₁- (identical to Ac-(Ac-Lys-Lys)Lys-), wherein Lys₁ is the first lysine residue, which is acetylated, covalently linked to the N-terminus of a peptide such as exendin-4 peptide, and (Ac-Lys-Lys) is the molecule covalently linked to the ε-amino group of said first lysine residue Lys₁. FIG. 1 illustrates this formula/structure.

In one embodiment Ac-(Ac-Lys-Lys)Lys- is covalently linked to the N-terminal of the exendin-4 peptide and/or to the side chain amino group of an amino alkyl amino acid residue within said exendin-4 peptide.

In one embodiment the branched amino acid probe comprises or consists of the formula: Ac-(Ac-Lys)Lys₁-.

In one embodiment the branched amino acid probe comprises or consists of the formula: (Ac-Lys-Lys)Lys₁-NH₂ (identical to (Ac-Lys-Lys)Lys-NH₂), wherein Lys₁ is the first lysine residue, which is amidated (—NH₂) at the C-terminal, and (Ac-Lys-Lys) is the molecule attached to the ε-amino group of said first lysine residue Lys₁. In one embodiment (Ac-Lys-Lys)Lys₁-NH₂ is attached to the C-terminal of the exendin-4 peptide.

In one embodiment the branched amino acid probe comprises or consists of a formula selected from the group consisting of (AAA)AAA₁-, (AAA-AAA)AAA₁-, (AAA-AAA-AAA)AAA₁-, (AAA-AAA-AAA-AAA)AAA₁-, (AAA-Gly-AAA)AAA₁-, (AAA-AAA-Gly)AAA₁-, (AAA-Gly)AAA₁-, (AAA-Ala-AAA)AAA₁-, (AAA-AAA-Ala)AAA₁-, (AAA-Ala)AAA₁-, (AAA-His-AAA)AAA₁-, (AAA-AAA-His)AAA₁-, (AAA-His)AAA₁-, (AAA-Arg-AAA)AAA₁-, (AAA-AAA-Arg)AAA₁-, and (AAA-Arg)AAA₁-. In one embodiment said first amino alkyl amino acid reside (AAA₁-) is N-terminally acetylated or C-terminally amidated.

In one embodiment the branched amino acid probe comprises or consists of a formula selected from the group consisting of (Lys)Lys₁-, (Lys-Lys)Lys₁-, (Lys-Lys-Lys)Lys₁-, (Lys-Lys-Lys-Lys)Lys₁-, (Lys-Gly-Lys)Lys₁-, (Lys-Lys-Gly)Lys₁-, (Lys-Gly)Lys₁-, (Lys-Ala-Lys)Lys₁-, (Lys-Lys-Ala)Lys₁-, (Lys-Ala)Lys₁-, (Lys-His-Lys)Lys₁-, (Lys-Lys-His)Lys₁-, (Lys-His)Lys₁-, (Lys-Arg-Lys)Lys₁-, (Lys-Lys-Arg)Lys₁-, and (Lys-Arg)Lys₁-. In one embodiment said first lysine reside (Lys₁-) is N-terminally acetylated or C-terminally amidated.

In one embodiment the branched amino acid probe comprises or consists of a formula selected from the group consisting of Ac-(Ac-Lys)Lys₁-, Ac-(Ac-Lys-Lys)Lys₁-, Ac-(Ac-Lys-Lys-Lys)Lys₁-, Ac-(Ac-Lys-Lys-Lys-Lys)Lys₁-, Ac-(Ac-Lys-Gly-Lys)Lys₁-, Ac-(Ac-Lys-Lys-Gly)Lys₁-, Ac-(Ac-Lys-Gly)Lys₁-, Ac-(Ac-Lys-Ala-Lys)Lys₁-, Ac-(Ac-Lys-Lys-Ala)Lys₁-, Ac-(Ac-Lys-Ala)Lys₁-, Ac-(Ac-Lys-His-Lys)Lys₁-, Ac-(Ac-Lys-Lys-His)Lys₁-, Ac-(Ac-Lys-His)Lys₁-, Ac-(Ac-Lys-Arg-Lys)Lys₁-, Ac-(Ac-Lys-Lys-Arg)Lys₁-, and Ac-(Ac-Lys-Arg)Lys₁-.

In one embodiment the branched amino acid probe comprises or consists of a formula selected from the group consisting of (Ac-Lys)Lys₁-NH₂, (Ac-Lys-Lys)Lys₁-NH₂, (Ac-Lys-Lys-Lys)Lys₁-NH₂, (Ac-Lys-Lys-Lys-Lys)Lys₁-NH₂, (Ac-Lys-Gly-Lys)Lys₁-NH₂, (Ac-Lys-Lys-Gly)Lys₁-NH₂, (Ac-Lys-Gly)Lys₁-NH₂, (Ac-Lys-Ala-Lys)Lys₁-NH₂, (Ac-Lys-Lys-Ala)Lys₁-NH₂, (Ac-Lys-Ala)Lys₁-NH₂, (Ac-Lys-His-Lys)Lys₁-NH₂, (Ac-Lys-Lys-His)Lys₁NH₂, (Ac-Lys-His) Lys₁-NH₂, (Ac-Lys-Arg-Lys)Lys₁-NH₂, (Ac-Lys-Lys-Arg) Lys₁-NH₂, and (Ac-Lys-Arg)Lys₁-NH₂.

More specifically, in one embodiment the branched amino acid probe comprises or consists of a formula selected from the group consisting of Ac-(Ac-Lys)Lys₁-, Ac-(Ac-Lys-Lys)Lys₁-, Ac-(Ac-Lys-Lys-Lys)Lys₁-, Ac-(Ac-Lys-Lys-Lys-Lys)Lys₁-, Ac-(Ac-Lys-Gly-Lys)Lys₁-, Ac-(Ac-Lys-Lys-Gly)Lys₁- and Ac-(Ac-Lys-Gly)Lys₁-.

In one embodiment the branched amino acid probe comprises or consists of the formula: Ac-(Ac-Lys)Lys₂-Lys₁-, wherein Lys₁ is the first lysine residue, Lys₂ is the second lysine residue which is acetylated and covalently linked to Lys₁ via a peptide bond, and (Ac-Lys) is the molecule covalently linked to the ε-amino group of said second lysine residue Lys₂.

In one embodiment the branched amino acid probe comprises or consists of the formula: Ac-Lys₂-(Ac-Lys)Lys₁-, wherein the molecule (Ac-Lys) is covalently linked to the ε-amino group of said first lysine residue Lys₁.

In one embodiment the branched amino acid probe(s) is selected from the group consisting of

Ac-(Ac-Lys)Lys-Lys-, (Ac-Lys)Lys-Lys-, Ac-(Lys)Lys-Lys-, (Lys)Lys-Lys-, (Ac-Lys)LysLys-NH₂, (Lys)Lys-Lys-NH₂;
Ac-Lys-(Ac-Lys)Lys-, Lys-(Ac-Lys)Lys-, Ac-Lys-(Lys)Lys-, Lys-(Lys)LysLys-(Ac-Lys)Lys-NH₂, Lys-(Lys) Lys-NH₂;
Ac-(Ac-Lys-Lys)-Lys-, (Ac-Lys-Lys)-Lys-, Ac-(Lys-Lys)-Lys- and (Lys-Lys)-Lys(Ac-Lys-Lys)-Lys-NH₂, and (Lys-Lys)-Lys-NH₂.

In one embodiment the branched amino acid probe(s) is selected from the group consisting of Ac-(Ac-Lys)Lys-, Ac-(Lys)Lys-, (Ac-Lys)Lys-NH₂, (Lys)Lys-NH₂ and (Lys)Lys-.

In one embodiment the branched amino acid probe is selected from the group consisting of Ac-(Ac-Lys)Lys₂-Lys₁-, Ac-(Ac-Lys-Lys)Lys₂-Lys₁-, Ac-(Ac-Lys-Gly)Lys₂-Lys₁-, Ac-(Ac-Lys-Lys-Lys)Lys₂-Lys₁-, Ac-(Ac-Lys-Lys-Lys-Lys)Lys₂-Lys₁-, Ac-Lys₂-(Ac-Lys)-Lys₁-, Ac-Lyse-(Ac-Lys-Lys)-Lys₁-, Ac-Lyse-(Ac-Lys-Gly)-Lys₁-, Ac-Lyse-(Ac-LysLys-Lys)-Lys₁-, Ac-Lyse-(Ac-Lys-Lys-Lys-Lys)-Lys₁-, Ac-(Ac-Lys)Lys₂-(Ac-Lys₂-Lys₁-, Ac-(Ac-Lys)Lys₂-(Ac-Lys-Lys₂-Lys₁-, and Ac-(Ac-Lys-Lys)Lys₂-(Ac-Lys-Lys₂-Lys₁-.

More specifically, in one embodiment the branched amino acid probe is selected from the group consisting of Ac-(Ac-Lys)Lys₂-Lys₁-, Ac-(Ac-Lys-Lys)Lys₂-Lys₁-, Ac-(Ac-Lys-Gly)Lys₂-Lys₁-, Ac-Lyse-(Ac-Lys)-Lys₁-, Ac-Lyse-(Ac-Lys-Lys)-Lys₁-, Ac-Lyse-(Ac-Lys-Gly)-Lys₁-, Ac-(Ac-Lys)Lys₂-(Ac-Lys₂-Lys₁-, Ac-(Ac-Lys)Lys₂-(Ac-Lys-Lys₂-Lys₁-, and Ac-(Ac-Lys-Lys)Lys₂-(Ac-Lys-Lys₂-Lys₁-.

In one embodiment the branched amino acid probe is selected from the group consisting of Ac-Lys₃-Lys₂-(Ac-Lys)Lys₁-, Ac-Lys₃-(Ac-Lys)Lys₂-Lys₁-, Ac-(Ac-Lys)Lys₃-Lys₂-Lys₁-, Ac-Lys₃-(Ac-Lys)Lys₂-(Ac-Lys)Lys₁-, Ac-(Ac-Lys)Lys₃-(Ac-Lys)Lys₂-Lys₁-, and Ac-(Ac-Lys)Lys₃-Lys₂-(Ac-Lys)Lys₁-.

In a particular embodiment the branched amino acid probe is selected from the group consisting of Ac-(Ac-Lys)Lys₁-, Ac-(Ac-Lys-Lys)Lys₁-, Ac-(Ac-Lys-Lys-Lys)Lys₁-, Ac-(Ac-Lys-Lys-Lys-Lys)Lys₁-, Ac-(Ac-Lys-Gly-Lys)Lys₁-, Ac-(Ac-Lys-Lys-Gly)Lys₁-, Ac-(Ac-Lys-Gly)Lys₁-, Ac-(Ac-Lys)Lys₂-Lys₁-, Ac-(Ac-Lys-Lys)Lys₂-Lys₁-, Ac-(Ac-Lys-Gly)Lys₂-Lys₁-, Ac-Lyse-(Ac-Lys)-Lys₁-, Ac-Lyse-(Ac-Lys-Lys)-Lys₁-, Ac-Lys₂-(Ac-Lys-Gly)-Lys₁-, Ac-(Ac-Lys)Lys₂-(Ac-Lys₂-Lys₁-, Ac-(Ac-Lys)Lys₂-(Ac-Lys-Lys₂-Lys₁-, Ac-(Ac-Lys-Lys)Lys₂-(Ac-Lys-Lys₂-Lys₁-, Ac-Lys₃-Lys₂-(Ac-Lys)Lys₁-, Ac-Lys₃-(Ac-Lys)Lys₂-Lys₁-, Ac-(Ac-Lys)Lys₃-Lyse-Lys₁-, Ac-Lys₃-(Ac-Lys)Lys₂-(Ac-Lys)Lys₁-, Ac-(Ac-Lys)Lys₃-(Ac-Lys)Lys₂-Lys₁-, and Ac-(Ac-Lys)Lys₃-Lyse-(Ac-Lys)Lys₁-. In one embodiment said branched amino acid probe is covalently linked to the N-terminal of the exendin-4 peptide and/or to the side chain amino group of an amino alkyl amino acid residue within said exendin-4 peptide.

In a particular embodiment, the branched amino acid probe consists of 2 or 3 lysine residues (selected from Lys and D-Lys).

In a particular embodiment, the branched amino acid probe consists of 3 lysine residues. In another embodiment, the branched amino acid probe consists of 2 lysine residues.

In a particular embodiment, the branched amino acid probe consists of a first and a second lysine residue selected from Lys and D-Lys, wherein one or both of the first and second lysine residues are modified by attaching to the ε-amino group of said first and/or second lysine residue one lysine residue selected from Lys and D-Lys; wherein each of said lysine residues are optionally acetylated at the alpha amino group.

In a particular embodiment, the branched amino acid probe consists of a first lysine residue selected from Lys and D-Lys, wherein said first lysine residue is modified by attaching to the ε-amino group of said first lysine residue two lysine residues selected from Lys and D-Lys; wherein each of said lysine residues are optionally acetylated at the alpha amino group.

Linking the Branched Amino Acid Probes and the Exendin-4 Peptide

As disclosed herein, the first amino alkyl amino acid residue of each of the one or more branched amino acid probes is covalently linked to the N-terminus of an exendin-4 peptide, covalently linked to the C-terminus of an exendin-4 peptide, and/or covalently linked to the side chain amino group of an amino alkyl amino acid residue within an exendin-4 peptide.

Attaching one or more branched amino acid probes to an exendin-4 peptide yields an exendin-4 peptide/BAP-conjugate.

The term covalently linked to the N-terminus of said exendin-4 peptide means that the first amino alkyl amino acid residue of the branched amino acid probe is covalently linked to the alpha amino group of the most N-terminal amino acid residue of exendin-4 peptide.

The term covalently linked to the C-terminus of said exendin-4 peptide means that the alpha amino group of the first amino alkyl amino acid residue of the branched amino acid probe is covalently linked to the most C-terminal amino acid residue of exendin-4 peptide.

Furthermore, it is understood that a branched amino acid probe in one embodiment is covalently linked to the side chain amino group of an amino alkyl amino acid residue within said exendin-4 peptide.

In one particular embodiment said amino alkyl amino acid residue within said exendin-4 peptide sequence is selected from the group consisting of an ornithine residue and a lysine residue. In one particular embodiment said amino alkyl amino acid residue within said peptide sequence is a lysine residue.

In one embodiment the first amino alkyl amino acid residue of the branched amino acid probe is covalently linked to the δ-amino group of an ornithine residue within said exendin-4 peptide or the ε-amino group of a lysine residue within said exendin-4 peptide.

In one embodiment the first amino alkyl amino acid residue of the branched amino acid probe is covalently linked to the ε-amino group of a lysine residue within said exendin-4 peptide.

In one embodiment the first amino alkyl amino acid residue of the branched amino acid probe is covalently linked to the N-terminus of said exendin-4 peptide.

In one embodiment the first amino alkyl amino acid residue of the branched amino acid probe is covalently linked to the C-terminus of said exendin-4 peptide.

It is understood that an amino alkyl amino acid residue within said peptide sequence means that the amino alkyl amino acid residue does not form part of the branched amino acid probe per se, but is a residue occurring within the existing amino acid sequence of the exendin-4 peptide. Said amino alkyl amino acid residue can be positioned at any position of the exendin-4 peptide.

In one embodiment a branched amino acid probe is covalently linked to the side chain amino group of Lys at position 27 within said exendin-4 peptide (Lys²⁷).

In one embodiment a branched amino acid probe is covalently linked to the side chain amino group of Lys at position 12 within said exendin-4 peptide (Lys¹²).

In one embodiment a peptide analogue comprising one or more branched amino acid probes means that the peptide analogue in one embodiment comprises 1 branched amino acid probe, such as 2 branched amino acid probes, for example 3 branched amino acid probes, such as 4 branched amino acid probes, for example 5 branched amino acid probes, such as 6 branched amino acid probes.

In principle the peptide analogue can comprise any number of branched amino acid probes provided they can be covalently linked to the peptide (N-terminally, C-terminally and/or one or more amino alkyl amino acid residues within said exendin-4 peptide).

In one embodiment the exendin-4 peptide analogue comprises 1 branched amino acid probe.

In one embodiment the exendin-4 peptide analogue comprises 1 branched amino acid probe, which branched amino acid probe is covalently bound to the N-terminus of the exendin-4 peptide.

In one embodiment the exendin-4 peptide analogue comprises 1 branched amino acid probe, which branched amino acid probe is covalently bound to the C-terminus of the exendin-4 peptide.

In one embodiment the exendin-4 peptide analogue comprises 1 branched amino acid probe, which branched amino acid probe is covalently linked to the side chain amino group of an amino alkyl amino acid residue within said exendin-4 peptide.

In one embodiment the exendin-4 peptide analogue comprises more than one (two or more) branched amino acid probe(s). In the embodiments wherein the exendin-4 peptide analogue comprises more than one branched amino acid probe it is understood that the more than one branched amino acid probes may individually be the same (identical) or different (non-identical).

In one embodiment the exendin-4 peptide analogue comprises 2 branched amino acid probes.

In one embodiment the exendin-4 peptide analogue comprises 2 branched amino acid probes, wherein one branched amino acid probe is covalently bound to the N-terminus of the exendin-4 peptide and another branched amino acid probe is covalently bound to the C-terminus of the exendin-4 peptide.

In one embodiment the exendin-4 peptide analogue comprises 2 branched amino acid probes, wherein one branched amino acid probe is covalently bound to the N-terminus of the exendin-4 peptide and another branched amino acid probe is covalently linked to the side chain amino group of an amino alkyl amino acid residue within said exendin-4 peptide.

In one embodiment the exendin-4 peptide analogue comprises 2 branched amino acid probes, wherein one branched amino acid probe is covalently bound to the C-terminus of the exendin-4 peptide and another branched amino acid probe is covalently linked to the side chain amino group of an amino alkyl amino acid residue within said exendin-4 peptide.

In one embodiment the peptide analogue comprises 2 branched amino acid probes, wherein each of the two branched amino acid probes are covalently linked to the side chain amino group of different (or separate) amino alkyl amino acid residues within said exendin-4 peptide.

In one embodiment the exendin-4 peptide analogue comprises 3 branched amino acid probes.

In one embodiment the exendin-4 peptide analogue comprises 3 branched amino acid probes, wherein the first branched amino acid probe is covalently bound to the N-terminus of the exendin-4 peptide, the second branched amino acid probe is covalently bound to the C-terminus of the exendin-4 peptide and the third branched amino acid probe is covalently linked to the side chain amino group of an amino alkyl amino acid residue within said exendin-4 peptide.

In one embodiment the exendin-4 peptide analogue comprises 3 branched amino acid probes, wherein the first branched amino acid probe is covalently bound to the N-terminus of the exendin-4 peptide, and the second and third branched amino acid probes are each covalently linked to the side chain amino group of different amino alkyl amino acid residues within said exendin-4 peptide.

In one embodiment the exendin-4 peptide analogue comprises 3 branched amino acid probes, wherein the first branched amino acid probe is covalently bound to the C-terminus of the exendin-4 peptide, and the second and third branched amino acid probes are each covalently linked to the side chain amino group of different amino alkyl amino acid residues within said exendin-4 peptide.

Exendin-4 Peptide

The exendin-4 peptide analogue as disclosed herein comprises an exendin-4 peptide and one or more branched amino acid probes, wherein said exendin-4 peptide is selected from the group consisting of des-Pro³⁸-exendin-4(1-39) (SEQ ID NO:1), des-Ser³⁹-exendin-4(1-39) (SEQ ID NO:2) and exendin-4(1-39) (SEQ ID NO:3) or a functional variant thereof.

In one embodiment the exendin-4 peptide is His-Gly-Glu-Gly-Thr-Phe-Thr-Ser-AspLeu-Ser-Lys-Gln-Met-Glu-Glu-Glu-Ala-Val-Arg-Leu-Phe-Ile-Glu-Trp-Leu-Lys-Asn-GlyGly-Pro-Ser-Ser-Gly-Ala-Pro-Pro-X₁ (SEQ ID NO:4), wherein X₁ is selected from the group consisting of Ser and Pro.

In one embodiment the exendin-4 peptide is des-Pro³⁸-exendin-4(1-39) (SEQ ID NO:1) or des-Ser³⁹-exendin-4(1-39) (SEQ ID NO:2).

In one embodiment the exendin-4 peptide is exendin-4(1-39) (SEQ ID NO:3) or a functional variant thereof.

In one embodiment the C-terminus of the exendin-4 peptide is a carboxylic acid, an aldehyde, an ester, or an amide, such as a primary amide (CONH₂) or a secondary amide. In one embodiment the C-terminus of the exendin-4 peptide is the unmodified C-terminal carboxylic group.

In one embodiment the exendin-4 peptide is C-terminally amidated (—NH₂). In one embodiment the exendin-4 peptide is not C-terminally amidated, in particular when a C-terminal branched amino acid probe is attached. In one embodiment the exendin-4 peptide is C-terminally amidated, in particular when a C-terminal branched amino acid probe is not attached.

In one embodiment the exendin-4 peptide is N-terminally acetylated (COCH₃ or Ac-). In one embodiment an exendin-4 peptide is not N-terminally acetylated, in particular when an N-terminal branched amino acid probe is attached. In one embodiment the exendin-4 peptide is N-terminally acetylated, in particular when an N-terminal branched amino acid probe is not attached.

In one embodiment the N-terminus of the exendin-4 peptide is the free amino moiety (H—). In one embodiment the N-terminal His of the exendin-4 peptide is the free amino moiety (H-His).

In one embodiment the N-terminal His is acetylated.

In one embodiment the C-terminus is amidated. In one embodiment the C-terminal Ser is amidated. In one embodiment the C-terminal Pro is amidated.

As used herein, each amino acid of the exendin-4(1-39) peptide (SEQ ID NO:3) from the N-terminus to the C-terminus is referred to as position 1, position 2, position 3 and so forth until position 39.

In one embodiment a functional variant of an exendin-4 peptide is a variant having one or more amino acid substitutions. In one embodiment a functional variant of an exendin-4 peptide is a variant having one or more conservative amino acid substitutions.

In one embodiment a functional variant of an exendin-4 peptide is a variant having one amino acid substitution. One amino acid substitution means that the amino acid differs between the original sequence and the variant sequence at one position.

In one embodiment a functional variant of an exendin-4 peptide is a variant having two amino acid substitutions. In one embodiment a functional variant of an exendin-4 peptide is a variant having three amino acid substitutions. In one embodiment a functional variant of an exendin-4 peptide is a variant having four amino acid substitutions. In one embodiment a functional variant of an exendin-4 peptide is a variant having five amino acid substitutions.

A functional variant of an exendin-4 peptide as defined herein can in principle have one or more substitutions at one or more positions. Individual amino acid residues in the exendin-4 peptide can be substituted with any given proteinogenic or non-proteinogenic amino acid.

The genetic code specifies 20 standard amino acids naturally incorporated into polypeptides (proteinogenic): Ala, Arg, Asn, Asp, Cys, Gln, Glu, Gly, His, Ile, Leu, Lys, Met, Phe, Pro, Ser, Tyr, Thr, Trp, Val, and 2 which are incorporated into proteins by unique synthetic mechanisms: Sec (selenocysteine, or U) and Pyl (pyrrolysine, 0). These are all L-stereoisomers.

Aside from the 22 standard or natural amino acids, there are many other non-naturally occurring amino acids (non-proteinogenic or non-standard). They are either not found in proteins, or are not produced directly and in isolation by standard cellular machinery. Non-standard amino acids are usually formed through modifications to standard amino acids, such as post-translational modifications. Examples of unnatural amino acid residues are Abu, Aib, Nle (Norleucine), DOrn (D-ornithine, deguanylated arginine), Nal (beta-2-naphthyl-alanine), D-Nal (beta-2-naphthyl-D-alanine), DArg, DTrp, DPhe and DVal.

Any amino acids as defined herein may be in the L- or D-configuration. If nothing is specified, reference to the L-isomeric form is preferably meant.

The term peptide also embraces post-translational modifications introduced by chemical or enzyme-catalyzed reactions, as are known in the art. Such post-translational modifications can be introduced prior to partitioning, if desired. Also, functional equivalents may comprise chemical modifications such as ubiquitination, labeling (e.g., with radionuclides, various enzymes, etc.), pegylation (derivatization with polyethylene glycol), or by insertion (or substitution by chemical synthesis) of amino acids, which do not normally occur in human proteins.

Exendin-4 peptides with N-terminal alkylations and C-terminal esterifications are also encompassed within the present disclosure. Functional equivalents also comprise glycosylated and covalent or aggregative conjugates formed with the same molecules, including dimers or unrelated chemical moieties. Such functional equivalents are prepared by linkage of functionalities to groups which are found in a fragment including at any one or both of the N- and C-termini, by means known in the art.

In some embodiments, the exendin-4 peptides disclosed herein are modified by acetylation, such as N-terminal acetylation. In some embodiments the exendin-4 peptides disclosed herein are modified by C-terminal amidation.

A functional variant of an exendin-4 peptide means that the variant retains the function of the non-variant or original sequence, at least to some extent.

An exendin-4 peptide and a functional variant thereof as defined herein include any exendin-4 peptide which binds to and preferably activates GLP-1R.

A functional exendin-4 peptide in one embodiment is an exendin-4 peptide, or functional variant thereof, which is an agonist of GLP-1R.

The term “agonist” in the present context refers to an exendin-4 peptide as defined herein, capable of binding to, or in some embodiments, capable of binding to at least some extent and/or activating a receptor, or in some embodiments, activating a receptor to at least some extent. For example, a GLP-1R agonist is thus capable of binding to and/or activating the GLP-1R.

An agonist may be an agonist of several different types of receptors, and thus capable of binding and/or activating several different types of receptors. Said agonist can also be a selective agonist which only binds and activates one type of receptor. The term “antagonist” in the present context refers to a substance capable of inhibiting the effect of a receptor agonist.

Full agonists bind (have affinity for) and activate a receptor, displaying full efficacy at that receptor. “Partial agonists” in the present context are peptides able to bind and activate a given receptor, but having only partial efficacy at the receptor relative to a full agonist. Partial agonists can act as antagonists when competing with a full agonist for receptor occupancy and producing a net decrease in the receptor activation compared to the effects or activation observed with the full agonist alone.

“Selective agonists” in the present context are compounds which are selective and therefore predominantly bind and activates one type of receptor. Thus a selective GLP1R agonist is selective for the GLP-1R.

Exendin-4 peptides as defined herein are in one embodiment capable of binding and activating to some extent the GLP-1R. Affinity refers to the number and size of intermolecular forces between a peptide ligand and its receptor, and residence time of the ligand at its receptor binding site; and receptor activation efficacy refers to the ability of the peptide ligand to produce a biological response upon binding to the target receptor and the quantitative magnitude of this response. In some embodiments, such differences in affinity and receptor activation efficacy are determined by receptor binding/activation studies which are conventional in the art, for instance by generating EC₅₀ and Emax values for stimulation of ligand binding in cells expressing one or several types of receptors as mentioned herein, or on tissues expressing the different types of receptors. High affinity means that a lower concentration of a ligand is needed to obtain a binding of 50% of the receptors compared to ligand peptides which have lower affinity; high receptor activation efficacy means that a lower concentration of the peptide is needed to obtain a 50% receptor activation response (low EC₅₀ value), compared to peptides which have lower affinity and/or receptor activity efficacy (higher EC₅₀ value).

In a particular embodiment, a functional exendin-4 peptide, or a variant of an exendin-4 peptide, is an exendin-4 peptide which has binding affinity and/or receptor efficacy for GLP-1R. This may be tested using conventional methods, or as outlined in examples 2 and 3.

In one particular embodiment, the exendin-4 peptides are capable of binding to and activating at least GLP-1R. In a further embodiment said peptide is a full agonist of GLP1R.

In one embodiment an exendin-4 peptide as defined herein, including functional variants thereof, is capable of one or more of
enhancing/stimulating glucose-dependent insulin secretion by pancreatic beta-bells,
augmenting pancreatic response (i.e. increases insulin secretion) in response to eating meals; the result is the release of a higher, more appropriate amount of insulin that helps lower the rise in blood sugar from eating,
lowering blood glucose in mammal,
increasing the number of beta cells in the pancreas
suppresses pancreatic release of glucagon in response to eating, which helps stop the liver from overproducing sugar when it is unneeded, which prevents hyperglycemia,
slowing down gastric emptying and thus decreasing the rate at which meal-derived glucose appears in the bloodstream,
reducing appetite, promoting satiety via hypothalamic receptors, and/or
reducing liver fat content.

Methods of Preparation

The exendin-4 peptide analogues as disclosed herein may be prepared by any suitable methods known in the art. Thus, in some embodiments the exendin-4 peptide and the branched amino acid probe are each prepared by standard peptide-preparation techniques, such as solution synthesis or solid phase peptide synthesis (SPPS) such as Merrifield-type solid phase synthesis.

The exendin-4 peptide analogues are in one embodiment prepared by solid phase synthesis by first constructing the pharmacologically active exendin-4 peptide sequence, using well-known standard protection, coupling and de-protection procedures, thereafter sequentially coupling the branched amino acid probe onto the active exendin-4 peptide in a manner similar to the construction of the active exendin-4 peptide, and finally cleaving off the entire exendin-4 peptide analogue from the carrier. This strategy yields an exendin-4 peptide, wherein the branched amino acid probe is covalently bound to the pharmacologically active exendin-4 peptide at the N-terminal nitrogen atom of the exendin-4 peptide.

In one embodiment, the alpha nitrogen on the final amino acid in the branched amino acid sequence is capped with acetyl, using standard acylation techniques, prior to or after coupling of the branched amino acid sequence on the active exendin-4 peptide.

Reactive moieties at the N- and C-termini, which facilitates amino acid coupling during synthesis, as well as reactive side chain functional groups, can interact with free termini or other side chain groups during synthesis and peptide elongation and negatively influence yield and purity. Chemical groups are thus developed that bind to specific amino acid functional groups and block, or protect, the functional group from nonspecific reactions. Purified, individual amino acids are reacted with these protecting groups prior to synthesis and then selectively removed during specific steps of peptide synthesis. Examples of N-terminal protecting groups are t-Boc and Fmoc, commonly used in solid-phase peptide synthesis. C-terminal protecting groups are mostly used in liquid-phase synthesis. Because N-terminal deprotection occurs continuously during peptide synthesis, protecting schemes have been established in which the different types of side chain protecting groups (benzyl; Bzl or tert-butyl; tBu) are matched to either Boc or Fmoc, respectively, for optimized deprotection.

In a particular embodiment, when preparing the branched amino acid probe, exemplified by Ac(Ac-Lys-Lys)Lys-, the protection group for Lys is Mtt, which as Fmoc protected amino acid is commercially available (Fmoc-Lys(Mtt)-OH; N-α-Fmoc-N-ε-4-methyltrityl-L-lysine, CAS #167393-62-6). Lys(Mtt) allows for capping Lys with acetyl or extending the sequence at the alpha amino group of lysine as it is not cleaved under the conditions that cleave Fmoc, and may be removed without cleavage of other side chain protection groups.

In a particular embodiment, when preparing the branched amino acid probe, exemplifled by (Ac-Lys-Lys)Lys-NH₂, the protection group for Lys is ivDde, which as Fmoc protected amino acid is commercially available (Fmoc-Lys(ivDde)-OH; N-α-Fmoc-N-ε-1-(4,4-dimethyl-2,6-dioxocyclohex-1-ylidene)-3-methylbutyl-L-lysine, CAS #204777-78-6). Lys(ivDde) allows for extending the sequence at the alpha amino group of lysine or capping Lys with acetyl as it is not cleaved under the conditions that cleave Fmoc, and may be removed without cleavage of other side chain protection groups.

The method of preparation is in some embodiments optimized by routine methods in the art that may increase the yield and/or quality of the thus prepared synthetic exendin-4 peptide. For instance, use of pseudoproline (oxazolidine) dipeptides in the Fmoc SPPS of serine- and threonine-containing peptides may lead to improvements in quality and yield of crude products and may help avoid unnecessary repeat synthesis of failed sequences. These dipeptides are easy to use: simply substitute a serine or threonine residue together with the preceding amino acid residue in the peptide sequence with the appropriate pseudoproline dipeptide. The native sequence is regenerated on cleavage and deprotection.

In one embodiment the sequence of the pharmacologically active exendin-4 peptide and the branched amino acid probe (or parts thereof) are each prepared separately by for example solution synthesis, solid phase synthesis, recombinant techniques, or enzymatic synthesis, followed by coupling of the (at least) two sequences by well-known segment condensation procedures, either in solution or using solid phase techniques, or a combination thereof.

In one embodiment, the exendin-4 peptide is prepared by recombinant DNA methods and the branched amino acid probe is prepared by solid or solution phase synthesis. The conjugation of the exendin-4 peptide and the branched amino acid probe is in one embodiment carried out by using chemical ligation. This technique allows for the assembling of totally unprotected peptide segments in a highly specific manner. In another embodiment, the conjugation is performed by protease-catalysed peptide bond formation, which offers a highly specific technique to combine totally unprotected peptide segments via a peptide bond.

In one embodiment, the C-terminal amino acid of the branched amino acid probe or the C-terminal amino acid of the exendin-4 peptide is covalently linked to the solid support material by means of a common linker such as 2,4-dimethoxy-4′-hydroxy-benzophenone, 4-(4-hydroxy-methyl-3-methoxyphenoxy)-butyric acid, 4-hydroxy-methylbenzoic acid, 4-hydroxymethyl-phenoxyacetic acid, 3-(4-hydroxymethylphenoxy)propionic acid, or p-{(R,S)-α-[1-(9H-Fluoren-9-yl)-methoxyformamido]-2,4-dimethoxyben-zyl}-phenoxyacetic acid (Rink amide linker).

Examples of suitable solid support materials (SSM) are e.g., functionalised resins such as polystyrene, polyacrylamide, polydimethylacrylamide, polyethyleneglycol, cellulose, polyethylene, polyethyleneglycol grafted on polystyrene, latex, dynabeads, etc.

The produced exendin-4 peptide analogues are in some embodiment cleaved from the solid support material by means of an acid such as trifluoracetic acid, trifluoromethanesulfonic acid, hydrogen bromide, hydrogen chloride, hydrogen fluoride, etc. optionally in combination with one phenol, thioanisole, etc., or the peptide conjugate are in other embodiments cleaved from the solid support by means of a base such as ammonia, hydrazine, an alkoxide, such as sodium ethoxide, an hydroxide, such as sodium hydroxide, etc.

In one embodiment the produced exendin-4 peptide analogues are isolated as salts, such as an acetate salt or maleate salt or any other salt known to the skilled person.

In other embodiments, the exendin-4 peptide analogues may be prepared or produced by recombinant techniques. Thus, in one aspect the peptide is produced by host cells comprising a first nucleic acid sequence encoding the exendin-4 peptide or exendin-4 peptide analogue operably associated with a second nucleic acid capable of directing expression in said host cells. In some embodiments the second nucleic acid sequence comprises or even consists of a promoter that will direct the expression of protein of interest in said cells. A skilled person will be readily capable of identifying useful second nucleic acid sequences (e.g. vectors and plasmids) for use in a given host cell.

The process of producing a recombinant peptide in general comprises the steps of: providing a host cell, preparing a gene expression construct comprising a first nucleic acid encoding the peptide operably linked to a second nucleic acid capable of directing expression of said protein of interest in the host cell, transforming the host cell with the construct and cultivating the host cell, thereby obtaining expression of the peptide. In one embodiment, the recombinantly produced peptide is excreted by the host cells. The host cell include any suitable host cell known in the art, including prokaryotic cells, yeast cells, insect cells and mammalian cells.

In one embodiment, the recombinant peptide thus produced is isolated by any conventional method and may be linked via conventional peptide bond forming chemistry to any suitably protected branched amino peptide moiety. The skilled person will be able to identify suitable protein isolation steps for purifying the peptide.

Methods of Treatment

It is an aspect to provide exendin-4 peptide analogues as defined herein for use as a medicament.

In another aspect, the present invention provides methods for treatment, prevention or alleviation of a medical condition. Such methods in one embodiment comprise one or more steps of administration or release of an effective amount of an exendin-4 peptide analogue, or a pharmaceutical composition comprising one or more such exendin-4 peptide analogues, to an individual in need thereof. In one embodiment, such steps of administration or release according to the present invention are simultaneous, sequential or separate.

An individual in need as referred to herein, is in one embodiment an individual that benefits from the administration of an exendin-4 peptide analogue or pharmaceutical composition according to the present invention. Such an individual in one embodiment suffers from a disease or condition or is at risk of suffering therefrom. The individual is in one embodiment any human being, male or female, infant, middle-aged or old. The disorder to be treated or prevented in the individual in one embodiment relates to the age of the individual, the general health of the individual, the medications used for treating the individual and whether or not the individual has a prior history of suffering from diseases or disorders that may have or have induced the condition in the individual.

The terms “treatment” and “treating” as used herein refer to the management and care of a patient for the purpose of combating a condition, disease or disorder. The term is intended to include the full spectrum of treatments for a given condition from which the patient is suffering, such as administration of the exendin-4 peptide analogue for the purpose of: alleviating or relieving symptoms or complications; delaying the progression of the condition, partially arresting the clinical manifestations, disease or disorder; curing or eliminating the condition, disease or disorder; and/or preventing or reducing the risk of acquiring the condition, disease or disorder, wherein “preventing” or “prevention” is to be understood to refer to the management and care of a patient for the purpose of hindering the development of the condition, disease or disorder, and includes the administration of the active compounds to prevent or reduce the risk of the onset of symptoms or complications. The patient to be treated is preferably a mammal, in particular a human being.

Medical Indications

The invention is in one embodiment directed to an exendin-4 peptide analogue as disclosed herein for use in the treatment of an ischemic condition, an inflammatory condition, an infection and/or a metabolic condition.

The invention is in one embodiment directed to use of an exendin-4 peptide analogue as disclosed herein for the manufacture of a medicament for the treatment of an ischemic condition, an inflammatory condition, an infection and/or a metabolic condition.

The invention is in one embodiment directed to a method for treatment of an ischemic condition, an inflammatory condition, an infection and/or a metabolic condition, said method comprising administering an effective amount of an exendin-4 peptide analogue to an individual in need thereof.

In one embodiment there is provided an exendin-4 peptide analogue as disclosed herein for use in the treatment of an ischemic and/or inflammatory condition in the tissue of one or more organs of a mammal.

In one embodiment, the ischemic and/or inflammatory condition in the tissue of one or more organs is an acute, subacute or chronic condition. In a further embodiment, the ischemic condition in the tissue of one or more organs is secondary ischemia.

In one embodiment said ischemic and/or inflammatory condition in the tissue of one or more organs is due to (or caused by) a condition selected from stroke, injury, septic shock, systemic hypotension, cardiac arrest due to heart attack, cardiac arrhythmia, atheromatous disease with thrombosis, embolism from the heart or from blood vessel from any organ, vasospasm, aortic aneurysm or aneurisms in other organs, coronary stenosis, myocardial infarction, angina pectoris, pericarditis, myocarditis, myxodemia, or endocarditis.

In a particular embodiment, said ischemic and/or inflammatory condition in the tissue of one or more organs is associated with reperfusion injury. Reperfusion injury is tissue damage caused when blood supply returns to the tissue after a period of ischemia or lack of oxygen.

In one embodiment said ischemic and/or inflammatory condition is associated with renal injuries, such as acute kidney injury (AKI), neprotoxicity and/or chronic renal failure (CRF).

In one embodiment said ischemic and/or inflammatory condition is associated with liver injuries.

In one embodiment there is provided an exendin-4 peptide analogue as disclosed herein for use in the treatment of diabetes mellitus type 2.

In one embodiment there is provided an exendin-4 peptide analogue as disclosed herein for use in the treatment of obesity.

In one embodiment there is provided an exendin-4 peptide analogue as disclosed herein for use in inducing/promoting/enhancing/satiety and/or the feeling of satiety; and/or reducing appetite.

In one embodiment there is provided an exendin-4 peptide analogue as disclosed herein for use in a method of one or more of
glycemic control,
lowering blood glucose,
stimulating glucose-dependent insulin secretion by pancreatic beta-bells,
increasing insulin secretion in response to eating, and/or
suppressing release of glucagon in response to eating.

The invention is in one embodiment directed to use of an exendin-4 peptide analogue for the manufacture of a medicament for the treatment of diabetes mellitus type 2 and/or obesity and/or promoting satiety and/or glycemic control.

The invention is in one embodiment directed to a method for treatment of diabetes mellitus type 2 and/or obesity and/or promoting satiety and/or glycemic control, said method comprising administering an exendin-4 peptide analogue to an individual in need thereof, such as administering a therapeutically effective amount of an exendin-4 peptide analogue.

In one embodiment said treatment is prophylactic, ameliorative and/or curative. In one embodiment, said mammal is a human (Homo sapiens).

Further Active Ingredients

In some embodiments, the exendin-4 peptide analogues as disclosed herein are combined with or comprise one or more further active ingredients which are understood as other therapeutic compounds or pharmaceutically acceptable derivatives thereof.

Methods for treatment according to the present invention in one embodiment thus further comprise one or more steps of administration of one or more further active ingredients, either concomitantly or sequentially, and in any suitable ratios.

Methods of treatment according to the present invention in one embodiment include a step wherein the pharmaceutical composition or exendin-4 peptide analogue as defined herein is administered simultaneously, sequentially or separately in combination with one or more further active ingredients.

In a particular embodiment the exendin-4 peptide analogues are administered in combination with, and/or formulated as a combination product, one or more further active ingredients selected from an oral glucose-lowering compound and/or insulin.

Administration and Dosage

A composition comprising an exendin-4 peptide analogue as defined herein is in one embodiment administered to individuals in need thereof in pharmaceutically effective doses or a therapeutically effective amount.

A therapeutically effective amount of an exendin-4 peptide analogue is in one embodiment an amount sufficient to cure, prevent, reduce the risk of, alleviate or partially arrest the clinical manifestations of a given disease or disorder and its complications. The amount that is effective for a particular therapeutic purpose will depend on the severity and the sort of the disorder as well as on the weight and general state of the subject. An amount adequate to accomplish this is defined as a “therapeutically effective amount”.

In one embodiment, the composition is administered in doses of from 1 μg/day to 100 mg/day; such as from 1 μg/day to 10 μg/day, such as 10 μg/day to 100 μg/day, such as 100 μg/day to 250 μg/day, such as 250 μg/day to 500 μg/day, such as 500 μg/day to 750 μg/day, such as 750 μg/day to 1 mg/day, such as 1 mg/day to 2 mg/day, such as 2 mg/day to 5 mg/day, or such as 5 mg/day to 10 mg/day, such as 10 mg/day to 20 mg/day, such as 20 mg/day to 30 mg/day, such as 30 mg/day to 40 mg/day, such as 40 mg/day to 50 mg/day, such as 50 mg/day to 75 mg/day, or such as 75 mg/day to 100 mg/day.

In one embodiment, one single dose of the composition is administered and may comprise of from 1 μg/kg body weight to 100 mg/kg body weight; such as from 1 to 10 μg/kg body weight, such as 10 to 100 μg/day, such as 100 to 250 μg/kg body weight, such as 250 to 500 μg/kg body weight, such as 500 to 750 μg/kg body weight, such as 750 μg/kg body weight to 1 mg/kg body weight, such as 1 mg/kg body weight to 2 mg/kg body weight, such as 2 to 5 mg/kg body weight, such as 5 to 10 mg/kg body weight, such as 10 to 20 mg/kg body weight, such as 20 to 30 mg/kg body weight, such as 30 to 40 mg/kg body weight, such as 40 to 50 mg/kg body weight, such as 50 to 75 mg/kg body weight, or such as 75 to 100 mg/kg body weight.

In one embodiment, a dose is administered one or several times per day, such as from 1 to 6 times per day, such as from 1 to 5 times per day, such as from 1 to 4 times per day, such as from 1 to 3 times per day, such as from 1 to 2 times per day, such as from 2 to 4 times per day, such as from 2 to 3 times per day. In one embodiment, a dose is administered less than once a day, such as once every second day or once a week.

Routes of Administration

It will be appreciated that the preferred route of administration will depend on the general condition and age of the subject to be treated, the nature of the condition to be treated, the location of the tissue to be treated in the body and the active ingredient chosen.

Systemic Treatment

In one embodiment, the route of administration allows for introducing the peptide analogue into the blood stream to ultimately target the sites of desired action.

In one embodiment the routes of administration is any suitable routes, such as an enteral route (including the oral, rectal, nasal, pulmonary, buccal, sublingual, transdermal, intracisternal and intraperitoneal administration), and/or a parenteral route (including subcutaneous, intramuscular, intrathecal, intravenous and intradermal administration).

Appropriate dosage forms for such administration may be prepared by conventional techniques.

Parenteral Administration

Parenteral administration is any administration route not being the oral/enteral route whereby the medicament avoids first-pass degradation in the liver. Accordingly, parenteral administration includes any injections and infusions, for example bolus injection or continuous infusion, such as intravenous administration, intramuscular administration or subcutaneous administration. Furthermore, parenteral administration includes inhalations and topical administration.

Accordingly, the peptide analogue or composition is in one embodiment administered topically to cross any mucosal membrane of an animal to which the substance or peptide is to be given, e.g. in the nose, vagina, eye, mouth, genital tract, lungs, gastrointestinal tract, or rectum, for example the mucosa of the nose, or mouth, and accordingly, parenteral administration may also include buccal, sublingual, nasal, rectal, vaginal and intraperitoneal administration as well as pulmonal and bronchial administration by inhalation or installation. In some embodiments, the peptide analogue is administered topically to cross the skin.

In one embodiment, the intravenous, subcutaneous and intramuscular forms of parenteral administration are employed.

Local Treatment

In one embodiment, the peptide analogue or composition is used as a local treatment, i.e. is introduced directly to the site(s) of action. Accordingly, the peptide may be applied to the skin or mucosa directly, or the peptide may be injected into the site of action, for example into the diseased tissue or to an end artery leading directly to the diseased tissue.

Pharmaceutical Formulations

In one embodiment the exendin-4 peptide analogues or pharmaceutically acceptable derivatives thereof are administered alone or in combination with pharmaceutically acceptable carriers or excipients, in either single or multiple doses. The pharmaceutical compositions or peptides as defined herein may be formulated with pharmaceutically acceptable carriers or diluents as well as any other known adjuvants and excipients in accordance with conventional techniques, such as those disclosed in Remington: The Science and Practice of Pharmacy, 20^{th} Edition, Gennaro, Ed., Mack Publishing Co., Easton, PA, 2000.

The term “pharmaceutically acceptable derivative” in present context includes pharmaceutically acceptable salts, which indicate a salt which is not harmful to the patient. Such salts include pharmaceutically acceptable basic or acid addition salts as well as pharmaceutically acceptable metal salts, ammonium salts and alkylated ammonium salts. A pharmaceutically acceptable derivative further includes pharmaceutically acceptable esters, prodrugs, or other precursors of a compound which may be biologically metabolized into the active compound, or crystal forms of a compound.

The pharmaceutical composition or pharmaceutically acceptable composition may be specifically formulated for administration by any suitable route, such as an enteral route, the oral, rectal, nasal, pulmonary, buccal, sublingual, transdermal, intracisternal, intraperitoneal, and parenteral (including subcutaneous, intramuscular, intrathecal, intravenous and intradermal) route.

In an embodiment, the pharmaceutical compositions or exendin-4 peptide analogues are formulated for crossing the blood-brain-barrier. In another embodiment, the pharmaceutical compositions or exendin-4 peptide analogues are formulated for not crossing the blood-brain-barrier.

Pharmaceutical compositions for oral administration include solid dosage forms such as hard or soft capsules, tablets, troches, dragees, pills, lozenges, powders and granules. Where appropriate, they can be prepared with coatings such as enteric coatings, or they can be formulated so as to provide controlled release of the active ingredient, such as sustained or prolonged release, according to methods well known in the art. In the same solid dosage form two active ingredients may be combined so as to provide controlled release of one active ingredient and immediate release of another active ingredient.

Liquid dosage forms for oral administration include solutions, emulsions, aqueous or oily suspensions, syrups and elixirs.

Pharmaceutical compositions for parenteral administration include sterile aqueous and non-aqueous injectable solutions, dispersions, suspensions or emulsions, as well as sterile powders to be reconstituted in sterile injectable solutions or dispersions prior to use, and depot injectable formulations.

Other suitable administration forms include suppositories, sprays, ointments, cremes/lotions, gels, inhalants, dermal patches, implants, etc.

In one embodiment, an exendin-4 peptide analogue is generally utilized as the free substance or as a pharmaceutically derivative such as a pharmaceutically acceptable ester or such as a salt thereof. Examples of the latter are: an acid addition salt of a compound having a free base functionality, and a base addition salt of a compound having a free acid functionality. The term “pharmaceutically acceptable salt” refers to a non-toxic salt of an exendin-4 peptide analogue as defined herein, which salts are generally prepared by reacting a free base with a suitable organic or inorganic acid, or by reacting an acid with a suitable organic or inorganic base. When an exendin-4 peptide analogue contains a free base functionality, such salts are prepared in a conventional manner by treating a solution or suspension of the compound with a chemical equivalent of a pharmaceutically acceptable acid. When an exendin-4 peptide analogue contains a free acid functionality, such salts are prepared in a conventional manner by treating a solution or suspension of the compound with a chemical equivalent of a pharmaceutically acceptable base. Physiologically acceptable salts of an exendin-4 peptide analogue with a hydroxy group include the anionic form of the compound in combination with a suitable cation, such as sodium or ammonium ion. Other salts which are not pharmaceutically acceptable may be useful in the preparation of exendin-4 peptide analogues, and these form a further aspect. Pharmaceutically acceptable acid addition salts include, but are not limited to, hydrochloride, hydrobromide, hydroiodide, nitrate, sulfate, bisulfate, phosphate, acid phosphate, isonicotinate, acetate, trifluoroacetate, trichloroacetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzensulfonate, p-toluenesulfonate and pamoate (i.e., 1,1′-methylene-bis-(2-hydroxy-3-naphthoate)) salts.

In one embodiment the exendin-4 peptide analogues are on crystalline forms, for example co-crystallized forms or hydrates of crystalline forms.

The term “prodrug” refers to peptides that are rapidly transformed in vivo to yield the parent compound of the above formulae, for example, by hydrolysis in blood or by metabolism in cells, such as for example the cells of the basal ganglia. A thorough discussion is provided in T. Higuchi and V Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987, both of which are hereby incorporated by reference. Examples of prodrugs include pharmaceutically acceptable, non-toxic esters of the compounds disclosed herein. Esters of the compounds may be prepared according to conventional methods “March's Advanced Organic Chemistry, 5^{th} Edition”. M. B. Smith & J. March, John Wiley & Sons, 2001.

In one embodiment, for parenteral administration, solutions of exendin-4 peptide analogues in sterile aqueous solution, in aqueous propylene glycol or in sesame or peanut oil are employed. Aqueous solutions should be suitably buffered where appropriate, and the liquid diluent rendered isotonic with, e.g., sufficient saline or glucose. Aqueous solutions are particularly suitable for intravenous, intramuscular, subcutaneous and intraperitoneal administration. The sterile aqueous media to be employed are all readily available by standard techniques known to those skilled in the art.

Suitable pharmaceutical carriers include inert solid diluents or fillers, sterile aqueous solutions and various organic solvents. Examples of solid carriers are lactose, terra alba, sucrose, cyclodextrin, talc, gelatine, agar, pectin, acacia, magnesium stearate, stearic acid and lower alkyl ethers of cellulose. Examples of liquid carriers are syrup, peanut oil, olive oil, phospholipids, fatty acids, fatty acid amines, polyoxyethylene and water. Moreover, the carrier or diluent may include any sustained release material known in the art, such as glyceryl monostearate or glyceryl distearate, alone or mixed with a wax. The pharmaceutical compositions formed by combining the present compounds and the pharmaceutically acceptable carriers are then readily administered in a variety of dosage forms suitable for the disclosed routes of administration. The formulations may conveniently be presented in unit dosage form by methods known in the art of pharmacy.

Formulations suitable for oral administration may be presented as discrete units, such as capsules or tablets, which each contain a predetermined amount of the active ingredient, and which may include a suitable excipient.

Furthermore, the orally available formulations may be in the form of a powder or granules, a solution or suspension in an aqueous or non-aqueous liquid, or an oil-in-water or water-in-oil liquid emulsion.

Compositions intended for oral use may be prepared according to any known method, and such compositions may contain one or more agents selected from the group consisting of sweetening agents, flavouring agents, colouring agents and preserving agents in order to provide pharmaceutically elegant and palatable preparations. Tablets may contain the active ingredient(s) in admixture with non-toxic pharmaceutically acceptable excipients which are suitable for the manufacture of tablets. These excipients may, for example, be: inert diluents, such as calcium carbonate, sodium carbonate, lactose, calcium phosphate or sodium phosphate; granulating and disintegrating agents, for example corn starch or alginic acid; binding agents, for example, starch, gelatine or acacia; and lubricating agents, for example magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated by the techniques described in U.S. Pat. Nos. 4,356,108; 4,166,452; and 4,265,874, the contents of which are incorporated herein by reference, to form osmotic therapeutic tablets for controlled release.

Formulations for oral use may also be presented as hard gelatine capsules where the active ingredient is mixed with an inert solid diluent, for example, calcium carbonate, calcium phosphate or kaolin, or a soft gelatine capsules wherein the active ingredient is mixed with water or an oil medium, for example peanut oil, liquid paraffin, or olive oil. Aqueous suspensions may contain the present compound in admixture with excipients suitable for the manufacture of aqueous suspensions. Such excipients are suspending agents, for example sodium carboxymethylcellulose, methylcellulose, hydroxypropylmethylcellulose, sodium alginate, polyvinylpyrrolidone, gum tragacanth and gum acacia; dispersing or wetting agents may be a naturally-occurring phosphatide such as lecithin, or condensation products of an alkylene oxide with fatty acids, for example polyoxyethylene stearate, or condensation products of ethylene oxide with long chain aliphatic alcohols, for example, heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more colouring agents, one or more flavouring agents, and one or more sweetening agents, such as sucrose or saccharin.

Oily suspensions may be formulated by suspending the active ingredient in a vegetable oil, for example arachis oil, olive oil, sesame oil or coconut oil, or in a mineral oil such as a liquid paraffin. The oily suspensions may contain a thickening agent, for example beeswax, hard paraffin or cetyl alcohol. Sweetening agents such as those set forth above, and flavouring agents may be added to provide a palatable oral preparation. These compositions may be preserved by the addition of an anti-oxidant such as ascorbic acid.

Dispersible powders and granules suitable for preparation of an aqueous suspension by the addition of water provide the active compound in admixture with a dispersing or wetting agent, suspending agent and one or more preservatives. Suitable dispersing or wetting agents and suspending agents are exemplified by those already mentioned above. Additional excipients, for example, sweetening, flavouring, and colouring agents may also be present.

The pharmaceutical compositions comprising exendin-4 peptide analogues may also be in the form of oil-in-water emulsions. The oily phase may be a vegetable oil, for example, olive oil or arachis oil, or a mineral oil, for example a liquid paraffin, or a mixture thereof. Suitable emulsifying agents may be naturally-occurring gums, for example gum acacia or gum tragacanth, naturally-occurring phosphatides, for example soy bean, lecithin, and esters or partial esters derived from fatty acids and hexitol anhydrides, for example sorbitan monooleate, and condensation products of said partial esters with ethylene oxide, for example polyoxyethylene sorbitan monooleate. The emulsions may also contain sweetening and flavouring agents.

Syrups and elixirs may be formulated with sweetening agents, for example glycerol, propylene glycol, sorbitol or sucrose. Such formulations may also contain a demulcent, a preservative and flavouring and colouring agent. The pharmaceutical compositions may be in the form of a sterile injectable aqueous or oleaginous suspension. This suspension may be formulated according to the known methods using suitable dispersing or wetting agents and suspending agents described above. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution, and isotonic sodium chloride solution. In addition, sterile, fixed oils are conveniently employed as solvent or suspending medium. For this purpose, any bland fixed oil may be employed using synthetic mono- or diglycerides. In addition, fatty acids such as oleic acid find use in the preparation of injectables.

The compositions may also be in the form of suppositories for rectal administration of the compounds. These compositions can be prepared by mixing the exendin-4 peptide analogues with a suitable non-irritating excipient which is solid at ordinary temperatures but liquid at the rectal temperature and will thus melt in the rectum to release the drug. Such materials include, for example, cocoa butter and polyethylene glycols.

Exendin-4 peptide analogues as disclosed herein may also be administered in the form of liposome delivery systems, such as small unilamellar vesicles, large unilamellar vesicles, and multilamellar vesicles. Liposomes may be formed from a variety of phospholipids, such as but not limited to cholesterol, stearylamine or phosphatidylcholines.

In addition, some exendin-4 peptide analogues as disclosed herein may form solvates with water or common organic solvents.

Thus, a further embodiment provides a pharmaceutical composition comprising an exendin-4 peptide analogue, or a pharmaceutically acceptable salt, solvate, or prodrug thereof, and one or more pharmaceutically acceptable carriers, excipients, or diluents.

### Examples of Sequences

| SEQ ID NO/ | |
|---|---|
| description | Sequence |
| SEQ ID NO: 1 | His-Gly-Glu-Gly-Thr-Phe-Thr-Ser- |
| des-Pro³⁸- | Asp-Leu-Ser-Lys-Gln-Met-Glu-Glu- |
| exendin-4 | Glu-Ala-Val-Arg-Leu-Phe-Ile-Glu- |
| (1-39) | Trp-Leu-Lys-Asn-Gly-Gly-Pro-Ser- |
| | Ser-Gly-Ala-Pro-Pro-Ser |
| SEQ ID NO: 2 | His-Gly-Glu-Gly-Thr-Phe-Thr-Ser- |
| des-Ser³⁹- | Asp-Leu-Ser-Lys-Gln-Met-Glu-Glu- |
| exendin-4 | Glu-Ala-Val-Arg-Leu-Phe-Ile-Glu- |
| | Trp-Leu-Lys-Asn-Gly-Gly-Pro-Ser- |
| | Ser-Gly-Ala-Pro-Pro-Pro |
| SEQ ID NO: 3 | His-Gly-Glu-Gly-Thr-Phe-Thr-Ser- |
| Exendin-4 | Asp-Leu-Ser-Lys-Gln-Met-Glu-Glu- |
| (1-39) | Glu-Ala-Val-Arg-Leu-Phe-Ile-Glu- |
| | Trp-Leu-Lys-Asn-Gly-Gly-Pro-Ser- |
| | Ser-Gly-Ala-Pro-Pro-Pro-Ser |
| SEQ ID NO: 4 | His-Gly-Glu-Gly-Thr-Phe-Thr-Ser- |
| des-Pro³⁸/ | Asp-Leu-Ser-Lys-Gln-Met-Glu-Glu- |
| des-Ser³⁹ | Glu-Ala-Val-Arg-Leu-Phe-Ile-Glu- |
| | Trp-Leu-Lys-Asn-Gly-Gly-Pro-Ser- |
| | Ser-Gly-Ala-Pro-Pro-X₁ |
| SEQ ID NO: 5 | His-Gly-Glu-Gly-Thr-Phe-Thr-Ser- |
| Lixisenatide | Asp-Leu-Ser-Lys-Gln-Met-Glu-Glu- |
| (Lyxumia): | Glu-Ala-Val-Arg-Leu-Phe-Ile-Glu- |
| des-Pro³⁸- | Trp-Leu-Lys-Asn-Gly-Gly-Pro-Ser- |
| Exendin-4- | Ser-Gly-Ala-Pro-Pro-Ser-(Lys)₆- |
| SIP | NH₂ |
| SEQ ID NO: 6 | His-Ala-Glu-Gly-Thr-Phe-Thr-Ser- |
| GLP-1 (7-36) | Asp-Val-Ser-Ser-Tyr-Leu-Glu-Gly- |
| | Gln-Ala-Ala-Lys-Glu-Phe-Ile-Ala- |
| | Trp-Leu-Val-Lys-Gly-Arg-Gly |

The below BAP′ed exendin-4-analogues are means to serve as non-limiting examples, of peptides having a 2-amino acid BAP, a 3-amino acid BAP or a 4-amino acid BAP attached to the N-terminus, the C-terminus, and/or within the sequence.

2-Amino Acid BAPs

Ac-(Ac-Lys)Lys-SEQ ID NO: 1:
Ac-(Ac-Lys)Lys-His-Gly-Glu-Gly-Thr-Phe-Thr-Ser-
Asp-Leu-Ser-Lys-Gln-Met-Glu-Glu-Glu-Ala-Val-Arg-
Leu-Phe-Ile-Glu-Trp-Leu-Lys-Asn-Gly-Gly-Pro-Ser-
Ser-Gly-Ala-Pro-Pro-Ser
Ac-(Ac-Lys)Lys-SEQ ID NO: 2
Ac-(Ac-Lys)Lys-His-Gly-Glu-Gly-Thr-Phe-Thr-Ser-
Asp-Leu-Ser-Lys-Gln-Met-Glu-Glu-Glu-Ala-Val-Arg-
Leu-Phe-Ile-Glu-Trp-Leu-Lys-Asn-Gly-Gly-Pro-Ser-
Ser-Gly-Ala-Pro-Pro-Pro
Ac-(Ac-Lys)Lys-SEQ ID NO: 3
Ac-(Ac-Lys)Lys-His-Gly-Glu-Gly-Thr-Phe-Thr-Ser-
Asp-Leu-Ser-Lys-Gln-Met-Glu-Glu-Glu-Ala-Val-Arg-
Leu-Phe-Ile-Glu-Trp-Leu-Lys-Asn-Gly-Gly-Pro-Ser-
Ser-Gly-Ala-Pro-Pro-Pro-Ser

The C-terminus may be amidated (—NH₂).

SEQ ID NO: 1-(Ac-Lys)Lys-NH₂:
His-Gly-Glu-Gly-Thr-Phe-Thr-Ser-Asp-Leu-Ser-Lys-
Gln-Met-Glu-Glu-Glu-Ala-Val-Arg-Leu-Phe-Ile-Glu-
Trp-Leu-Lys-Asn-Gly-Gly-Pro-Ser-Ser-Gly-Ala-Pro-
Pro-Ser-(Ac-Lys)Lys-NH₂
SEQ ID NO: 2-(Ac-Lys)Lys-NH₂:
His-Gly-Glu-Gly-Thr-Phe-Thr-Ser-Asp-Leu-Ser-Lys-
Gln-Met-Glu-Glu-Glu-Ala-Val-Arg-Leu-Phe-Ile-Glu-
Trp-Leu-Lys-Asn-Gly-Gly-Pro-Ser-Ser-Gly-Ala-Pro-
Pro-Pro-(Ac-Lys)Lys-NH₂
SEQ ID NO: 3-(Ac-Lys)Lys-NH₂:
His-Gly-Glu-Gly-Thr-Phe-Thr-Ser-Asp-Leu-Ser-Lys-
Gln-Met-Glu-Glu-Glu-Ala-Val-Arg-Leu-Phe-Ile-Glu-
Trp-Leu-Lys-Asn-Gly-Gly-Pro-Ser-Ser-Gly-Ala-Pro-
Pro-Pro-Ser-(Ac-Lys)Lys-NH₂

The N-terminus may be acetylated (Ac), or H—.

SEQ ID NO: 1-[(Ac-Lys)Lys-NH₂]_{Lys27}:
His-Gly-Glu-Gly-Thr-Phe-Thr-Ser-Asp-Leu-Ser-Lys-
Gln-Met-Glu-Glu-Glu-Ala-Val-Arg-Leu-Phe-Ile-Glu-
Trp-Leu-[(Ac-Lys)Lys]Lys-Asn-Gly-Gly-Pro-Ser-
Ser-Gly-Ala-Pro-Pro-Ser
SEQ ID NO: 2-[(Ac-Lys)Lys-NH₂]_{Lys27}:
His-Gly-Glu-Gly-Thr-Phe-Thr-Ser-Asp-Leu-Ser-Lys-
Gln-Met-Glu-Glu-Glu-Ala-Val-Arg-Leu-Phe-Ile-Glu-
Trp-Leu-[(Ac-Lys)Lys]Lys-Asn-Gly-Gly-Pro-Ser-
Ser-Gly-Ala-Pro-Pro-Pro
SEQ ID NO: 3-[(Ac-Lys)Lys-NH₂]_{Lys27}:
His-Gly-Glu-Gly-Thr-Phe-Thr-Ser-Asp-Leu-Ser-Lys-
Gln-Met-Glu-Glu-Glu-Ala-Val-Arg-Leu-Phe-Ile-Glu-
Trp-Leu-[(Ac-Lys)Lys]Lys-Asn-Gly-Gly-Pro-Ser-
Ser-Gly-Ala-Pro-Pro-Pro-Ser
SEQ ID NO: 1-[(Ac-Lys)Lys-NH₂]_{Lys12}:
His-Gly-Glu-Gly-Thr-Phe-Thr-Ser-Asp-Leu-Ser-
[(Ac-Lys)Lys]Lys-Gln-Met-Glu-Glu-Glu-Ala-Val-
Arg-Leu-Phe-Ile-Glu-Trp-Leu-Lys-Asn-Gly-Gly-Pro-
Ser-Ser-Gly-Ala-Pro-Pro-Ser
SEQ ID NO: 2-[(Ac-Lys)Lys-NH₂]_{Lys12}:
His-Gly-Glu-Gly-Thr-Phe-Thr-Ser-Asp-Leu-Ser-
[(Ac-Lys)Lys]Lys-Gln-Met-Glu-Glu-Glu-Ala-Val-
Arg-Leu-Phe-Ile-Glu-Trp-Leu-Lys-Asn-Gly-Gly-Pro-
Ser-Ser-Gly-Ala-Pro-Pro-Pro
SEQ ID NO: 3-[(Ac-Lys)Lys-NH₂]_{Lys12}:
His-Gly-Glu-Gly-Thr-Phe-Thr-Ser-Asp-Leu-Ser-
[(Ac-Lys)Lys]Lys-Gln-Met-Glu-Glu-Glu-Ala-Val-
Arg-Leu-Phe-Ile-Glu-Trp-Leu-Lys-Asn-Gly-Gly-Pro-
Ser-Ser-Gly-Ala-Pro-Pro-Pro-Ser

The N-terminus may be acetylated (Ac), or H—, and the C-terminus may be amidated (NH₂).

3-Amino Acid BAPs

Ac-(Ac-Lys-Lys)Lys-SEQ ID NO: 1:
Ac-(Ac-Lys-Lys)Lys-His-Gly-Glu-Gly-Thr-Phe-Thr-
Ser-Asp-Leu-Ser-Lys-Gln-Met-Glu-Glu-Glu-Ala-Val-
Arg-Leu-Phe-Ile-Glu-Trp-Leu-Lys-Asn-Gly-Gly-Pro-
Ser-Ser-Gly-Ala-Pro-Pro-Ser
Ac-(Ac-Lys-Lys)Lys-SEQ ID NO: 2:
Ac-(Ac-Lys-Lys)Lys-His-Gly-Glu-Gly-Thr-Phe-Thr-
Ser-Asp-Leu-Ser-Lys-Gln-Met-Glu-Glu-Glu-Ala-Val-
Arg-Leu-Phe-Ile-Glu-Trp-Leu-Lys-Asn-Gly-Gly-Pro-
Ser-Ser-Gly-Ala-Pro-Pro-Pro
Ac-(Ac-Lys-Lys)Lys-SEQ ID NO: 3:
Ac-(Ac-Lys-Lys)Lys-His-Gly-Glu-Gly-Thr-Phe-Thr-
Ser-Asp-Leu-Ser-Lys-Gln-Met-Glu-Glu-Glu-Ala-Val-
Arg-Leu-Phe-Ile-Glu-Trp-Leu-Lys-Asn-Gly-Gly-Pro-
Ser-Ser-Gly-Ala-Pro-Pro-Pro-Ser
Ac-(Ac-Lys)Lys-Lys-SEQ ID NO: 1:
Ac-(Ac-Lys)Lys-Lys-His-Gly-Glu-Gly-Thr-Phe-Thr-
Ser-Asp-Leu-Ser-Lys-Gln-Met-Glu-Glu-Glu-Ala-Val-
Arg-Leu-Phe-Ile-Glu-Trp-Leu-Lys-Asn-Gly-Gly-Pro-
Ser-Ser-Gly-Ala-Pro-Pro-Ser
Ac-(Ac-Lys)Lys-Lys-SEQ ID NO: 2:
Ac-(Ac-Lys)Lys-Lys-His-Gly-Glu-Gly-Thr-Phe-Thr-
Ser-Asp-Leu-Ser-Lys-Gln-Met-Glu-Glu-Glu-Ala-Val-
Arg-Leu-Phe-Ile-Glu-Trp-Leu-Lys-Asn-Gly-Gly-Pro-
Ser-Ser-Gly-Ala-Pro-Pro-Pro
Ac-(Ac-Lys)Lys-Lys-SEQ ID NO: 3:
Ac-(Ac-Lys)Lys-Lys-His-Gly-Glu-Gly-Thr-Phe-Thr-
Ser-Asp-Leu-Ser-Lys-Gln-Met-Glu-Glu-Glu-Ala-Val-
Arg-Leu-Phe-Ile-Glu-Trp-Leu-Lys-Asn-Gly-Gly-Pro-
Ser-Ser-Gly-Ala-Pro-Pro-Pro-Ser
Ac-Lys-(Ac-Lys)Lys-SEQ ID NO: 1:
Ac-Lys-(Ac-Lys)Lys-His-Gly-Glu-Gly-Thr-Phe-Thr-
Ser-Asp-Leu-Ser-Lys-Gln-Met-Glu-Glu-Glu-Ala-Val-
Arg-Leu-Phe-Ile-Glu-Trp-Leu-Lys-Asn-Gly-Gly-Pro-
Ser-Ser-Gly-Ala-Pro-Pro-Ser
Ac-Lys-(Ac-Lys)Lys-SEQ ID NO: 2:
Ac-Lys-(Ac-Lys)Lys-His-Gly-Glu-Gly-Thr-Phe-Thr-
Ser-Asp-Leu-Ser-Lys-Gln-Met-Glu-Glu-Glu-Ala-Val-
Arg-Leu-Phe-Ile-Glu-Trp-Leu-Lys-Asn-Gly-Gly-Pro-
Ser-Ser-Gly-Ala-Pro-Pro-Pro
Ac-Lys-(Ac-Lys)Lys-SEQ ID NO: 3:
Ac-Lys-(Ac-Lys)Lys-His-Gly-Glu-Gly-Thr-Phe-Thr-
Ser-Asp-Leu-Ser-Lys-Gln-Met-Glu-Glu-Glu-Ala-Val-
Arg-Leu-Phe-Ile-Glu-Trp-Leu-Lys-Asn-Gly-Gly-Pro-
Ser-Ser-Gly-Ala-Pro-Pro-Pro-Ser

The C-terminus may be amidated (—NH₂).

SEQ ID NO: 1-(Ac-Lys-Lys)Lys:
His-Gly-Glu-Gly-Thr-Phe-Thr-Ser-Asp-Leu-Ser-Lys-
Gln-Met-Glu-Glu-Glu-Ala-Val-Arg-Leu-Phe-Ile-Glu-
Trp-Leu-Lys-Asn-Gly-Gly-Pro-Ser-Ser-Gly-Ala-Pro-
Pro-Ser-(Ac-Lys-Lys)Lys
SEQ ID NO: 2-(Ac-Lys-Lys)Lys:
His-Gly-Glu-Gly-Thr-Phe-Thr-Ser-Asp-Leu-Ser-Lys-
Gln-Met-Glu-Glu-Glu-Ala-Val-Arg-Leu-Phe-Ile-Glu-
Trp-Leu-Lys-Asn-Gly-Gly-Pro-Ser-Ser-Gly-Ala-Pro-
Pro-Pro-(Ac-Lys-Lys)Lys
SEQ ID NO: 3-(Ac-Lys-Lys)Lys:
His-Gly-Glu-Gly-Thr-Phe-Thr-Ser-Asp-Leu-Ser-Lys-
Gln-Met-Glu-Glu-Glu-Ala-Val-Arg-Leu-Phe-Ile-Glu-
Trp-Leu-Lys-Asn-Gly-Gly-Pro-Ser-Ser-Gly-Ala-Pro-
Pro-Pro-Ser-(Ac-Lys-Lys)Lys
SEQ ID NO: 1-(Ac-Lys)Lys-Lys-:
His-Gly-Glu-Gly-Thr-Phe-Thr-Ser-Asp-Leu-Ser-Lys-
Gln-Met-Glu-Glu-Glu-Ala-Val-Arg-Leu-Phe-Ile-Glu-
Trp-Leu-Lys-Asn-Gly-Gly-Pro-Ser-Ser-Gly-Ala-Pro-
Pro-Ser-(Ac-Lys)Lys-Lys
SEQ ID NO: 2-(Ac-Lys-Lys)Lys:
His-Gly-Glu-Gly-Thr-Phe-Thr-Ser-Asp-Leu-Ser-Lys-
Gln-Met-Glu-Glu-Glu-Ala-Val-Arg-Leu-Phe-Ile-Glu-
Trp-Leu-Lys-Asn-Gly-Gly-Pro-Ser-Ser-Gly-Ala-Pro-
Pro-Pro-(Ac-Lys)Lys-Lys
SEQ ID NO: 3-(Ac-Lys-Lys)Lys:
His-Gly-Glu-Gly-Thr-Phe-Thr-Ser-Asp-Leu-Ser-Lys-
Gln-Met-Glu-Glu-Glu-Ala-Val-Arg-Leu-Phe-Ile-Glu-
Trp-Leu-Lys-Asn-Gly-Gly-Pro-Ser-Ser-Gly-Ala-Pro-
Pro-Pro-Ser-(Ac-Lys)Lys-Lys
SEQ ID NO: 1-Lys-(Ac-Lys)Lys:
His-Gly-Glu-Gly-Thr-Phe-Thr-Ser-Asp-Leu-Ser-Lys-
Gln-Met-Glu-Glu-Glu-Ala-Val-Arg-Leu-Phe-Ile-Glu-
Trp-Leu-Lys-Asn-Gly-Gly-Pro-Ser-Ser-Gly-Ala-Pro-
Pro-Ser-Lys-(Ac-Lys)Lys
SEQ ID NO: 2-Lys-(Ac-Lys)Lys:
His-Gly-Glu-Gly-Thr-Phe-Thr-Ser-Asp-Leu-Ser-Lys-
Gln-Met-Glu-Glu-Glu-Ala-Val-Arg-Leu-Phe-Ile-Glu-
Trp-Leu-Lys-Asn-Gly-Gly-Pro-Ser-Ser-Gly-Ala-Pro-
Pro-Pro-Lys-(Ac-Lys)Lys
SEQ ID NO: 3-Lys-(Ac-Lys)Lys:
His-Gly-Glu-Gly-Thr-Phe-Thr-Ser-Asp-Leu-Ser-Lys-
Gln-Met-Glu-Glu-Glu-Ala-Val-Arg-Leu-Phe-Ile-Glu-
Trp-Leu-Lys-Asn-Gly-Gly-Pro-Ser-Ser-Gly-Ala-Pro-
Pro-Pro-Ser-Lys-(Ac-Lys)Lys

The N-terminus may be acetylated (Ac), and the C-terminal BAP may be amidated (NH₂).

SEQ ID NO: 1-(Ac-Lys-Lys)Lys:
His-Gly-Glu-Gly-Thr-Phe-Thr-Ser-Asp-Leu-Ser-Lys-
Gln-Met-Glu-Glu-Glu-Ala-Val-Arg-Leu-Phe-Ile-Glu-
Trp-Leu-Lys-Asn-Gly-Gly-Pro-Ser-Ser-Gly-Ala-Pro-
Pro-Ser-(Ac-Lys-Lys)Lys
SEQ ID NO: 2-(Ac-Lys-Lys)Lys:
His-Gly-Glu-Gly-Thr-Phe-Thr-Ser-Asp-Leu-Ser-Lys-
Gln-Met-Glu-Glu-Glu-Ala-Val-Arg-Leu-Phe-Ile-Glu-
Trp-Leu-Lys-Asn-Gly-Gly-Pro-Ser-Ser-Gly-Ala-Pro-
Pro-Pro-(Ac-Lys-Lys)Lys
SEQ ID NO: 3-(Ac-Lys-Lys)Lys:
His-Gly-Glu-Gly-Thr-Phe-Thr-Ser-Asp-Leu-Ser-Lys-
Gln-Met-Glu-Glu-Glu-Ala-Val-Arg-Leu-Phe-Ile-Glu-
Trp-Leu-Lys-Asn-Gly-Gly-Pro-Ser-Ser-Gly-Ala-Pro-
Pro-Pro-Ser-(Ac-Lys-Lys)Lys
SEQ ID NO: 1-(Ac-Lys)Lys-Lys-:
His-Gly-Glu-Gly-Thr-Phe-Thr-Ser-Asp-Leu-Ser-Lys-
Gln-Met-Glu-Glu-Glu-Ala-Val-Arg-Leu-Phe-Ile-Glu-
Trp-Leu-Lys-Asn-Gly-Gly-Pro-Ser-Ser-Gly-Ala-Pro-
Pro-Ser-(Ac-Lys)Lys-Lys
SEQ ID NO: 2-(Ac-Lys-Lys)Lys:
His-Gly-Glu-Gly-Thr-Phe-Thr-Ser-Asp-Leu-Ser-Lys-
Gln-Met-Glu-Glu-Glu-Ala-Val-Arg-Leu-Phe-Ile-Glu-
Trp-Leu-Lys-Asn-Gly-Gly-Pro-Ser-Ser-Gly-Ala-Pro-
Pro-Pro-(Ac-Lys)Lys-Lys
SEQ ID NO: 3-(Ac-Lys-Lys)Lys:
His-Gly-Glu-Gly-Thr-Phe-Thr-Ser-Asp-Leu-Ser-Lys-
Gln-Met-Glu-Glu-Glu-Ala-Val-Arg-Leu-Phe-Ile-Glu-
Trp-Leu-Lys-Asn-Gly-Gly-Pro-Ser-Ser-Gly-Ala-Pro-
Pro-Pro-Ser-(Ac-Lys)Lys-Lys
SEQ ID NO: 1-Lys-(Ac-Lys)Lys:
His-Gly-Glu-Gly-Thr-Phe-Thr-Ser-Asp-Leu-Ser-Lys-
Gln-Met-Glu-Glu-Glu-Ala-Val-Arg-Leu-Phe-Ile-Glu-
Trp-Leu-Lys-Asn-Gly-Gly-Pro-Ser-Ser-Gly-Ala-Pro-
Pro-Ser-Lys-(Ac-Lys)Lys
SEQ ID NO: 2-Lys-(Ac-Lys)Lys:
His-Gly-Glu-Gly-Thr-Phe-Thr-Ser-Asp-Leu-Ser-Lys-
Gln-Met-Glu-Glu-Glu-Ala-Val-Arg-Leu-Phe-Ile-Glu-
Trp-Leu-Lys-Asn-Gly-Gly-Pro-Ser-Ser-Gly-Ala-Pro-
Pro-Pro-Lys-(Ac-Lys)Lys
SEQ ID NO: 3-Lys-(Ac-Lys)Lys:
His-Gly-Glu-Gly-Thr-Phe-Thr-Ser-Asp-Leu-Ser-Lys-
Gln-Met-Glu-Glu-Glu-Ala-Val-Arg-Leu-Phe-Ile-Glu-
Trp-Leu-Lys-Asn-Gly-Gly-Pro-Ser-Ser-Gly-Ala-Pro-
Pro-Pro-Ser-Lys-(Ac-Lys)Lys

The N-terminus may be acetylated (Ac) or H— and the C-terminus may be amidated (NH₂).

SEQ ID NO: 1-[Ac-(Ac-Lys-Lys)Lys]_{Lys12}:
His-Gly-Glu-Gly-Thr-Phe-Thr-Ser-Asp-Leu-Ser-[Ac-
(Ac-Lys-Lys)Lys]Lys-Gln-Met-Glu-Glu-Glu-Ala-Val-
Arg-Leu-Phe-Ile-Glu-Trp-Leu-Lys-Asn-Gly-Gly-Pro-
Ser-Ser-Gly-Ala-Pro-Pro-Ser
SEQ ID NO: 2-[Ac-(Ac-Lys-Lys)Lys]_{Lys12}:
His-Gly-Glu-Gly-Thr-Phe-Thr-Ser-Asp-Leu-Ser-[Ac-
(Ac-Lys-Lys)Lys]Lys-Gln-Met-Glu-Glu-Glu-Ala-Val-
Arg-Leu-Phe-Ile-Glu-Trp-Leu-Lys-Asn-Gly-Gly-Pro-
Ser-Ser-Gly-Ala-Pro-Pro-Pro
SEQ ID NO: 3-[Ac-(Ac-Lys-Lys)Lys]_{Lys12}:
His-Gly-Glu-Gly-Thr-Phe-Thr-Ser-Asp-Leu-Ser-[Ac-
(Ac-Lys-Lys)Lys]Lys-Gln-Met-Glu-Glu-Glu-Ala-Val-
Arg-Leu-Phe-Ile-Glu-Trp-Leu-Lys-Asn-Gly-Gly-Pro-
Ser-Ser-Gly-Ala-Pro-Pro-Pro-Ser
SEQ ID NO: 1-[Ac-(Ac-Lys-Lys)Lys]_{Lys27}:
His-Gly-Glu-Gly-Thr-Phe-Thr-Ser-Asp-Leu-Ser-Lys-
Gln-Met-Glu-Glu-Glu-Ala-Val-Arg-Leu-Phe-Ile-Glu-
Trp-Leu-[Ac-(Ac-Lys-Lys)Lys]Lys-Asn-Gly-Gly-Pro-
Ser-Ser-Gly-Ala-Pro-Pro-Ser
SEQ ID NO: 2-[Ac-(Ac-Lys-Lys)Lys]_{Lys27}:
His-Gly-Glu-Gly-Thr-Phe-Thr-Ser-Asp-Leu-Ser-Lys-
Gln-Met-Glu-Glu-Glu-Ala-Val-Arg-Leu-Phe-Ile-Glu-
Trp-Leu-[Ac-(Ac-Lys-Lys)Lys]Lys-Asn-Gly-Gly-Pro-
Ser-Ser-Gly-Ala-Pro-Pro-Pro
SEQ ID NO: 3-[Ac-(Ac-Lys-Lys)Lys]_{Lys27}:
His-Gly-Glu-Gly-Thr-Phe-Thr-Ser-Asp-Leu-Ser-Lys-
Gln-Met-Glu-Glu-Glu-Ala-Val-Arg-Leu-Phe-Ile-Glu-
Trp-Leu-[Ac-(Ac-Lys-Lys)Lys]Lys-Asn-Gly-Gly-Pro-
Ser-Ser-Gly-Ala-Pro-Pro-Pro-Ser

The N-terminus may be acetylated (Ac) or H— and the C-terminus may be amidated (NH₂).

4-Amino Acid BAPs

Ac-(Ac-Lys-Lys-Lys)Lys-SEQ ID NO: 1:
Ac-(Ac-Lys-Lys-Lys)Lys-His-Gly-Glu-Gly-Thr-Phe-
Thr-Ser-Asp-Leu-Ser-Lys-Gln-Met-Glu-Glu-Glu-Ala-
Val-Arg-Leu-Phe-Ile-Glu-Trp-Leu-Lys-Asn-Gly-Gly-
Pro-Ser-Ser-Gly-Ala-Pro-Pro-Ser
SEQ ID NO: 1-(Ac-Lys-Lys-Lys)Lys:
His-Gly-Glu-Gly-Thr-Phe-Thr-Ser-Asp-Leu-Ser-Lys-
Gln-Met-Glu-Glu-Glu-Ala-Val-Arg-Leu-Phe-Ile-Glu-
Trp-Leu-Lys-Asn-Gly-Gly-Pro-Ser-Ser-Gly-Ala-Pro-
Pro-Ser-(Ac-Lys-Lys-Lys)Lys
Ac-(Ac-Lys-Gly-Lys)Lys-SEQ ID NO: 1:
Ac-(Ac-Lys-Gly-Lys)Lys-His-Gly-Glu-Gly-Thr-Phe-
Thr-Ser-Asp-Leu-Ser-Lys-Gln-Met-Glu-Glu-Glu-Ala-
Val-Arg-Leu-Phe-Ile-Glu-Trp-Leu-Lys-Asn-Gly-Gly-
Pro-Ser-Ser-Gly-Ala-Pro-Pro-Ser
SEQ ID NO: 1-(Ac-Lys-Gly-Lys)Lys-:
His-Gly-Glu-Gly-Thr-Phe-Thr-Ser-Asp-Leu-Ser-Lys-
Gln-Met-Glu-Glu-Glu-Ala-Val-Arg-Leu-Phe-Ile-Glu-
Trp-Leu-Lys-Asn-Gly-Gly-Pro-Ser-Ser-Gly-Ala-Pro-
Pro-Ser-(Ac-Lys-Gly-Lys)Lys
Ac-(Ac-Lys-Lys)Lys-Lys-SEQ ID NO: 1:
Ac-(Ac-Lys-Lys)Lys-Lys-His-Gly-Glu-Gly-Thr-Phe-
Thr-Ser-Asp-Leu-Ser-Lys-Gln-Met-Glu-Glu-Glu-Ala-
Val-Arg-Leu-Phe-Ile-Glu-Trp-Leu-Lys-Asn-Gly-Gly-
Pro-Ser-Ser-Gly-Ala-Pro-Pro-Ser
SEQ ID NO: 1-(Ac-Lys-Lys)Lys-Lys-:
His-Gly-Glu-Gly-Thr-Phe-Thr-Ser-Asp-Leu-Ser-Lys-
Gln-Met-Glu-Glu-Glu-Ala-Val-Arg-Leu-Phe-Ile-Glu-
Trp-Leu-Lys-Asn-Gly-Gly-Pro-Ser-Ser-Gly-Ala-Pro-
Pro-Ser-(Ac-Lys-Lys)Lys-Lys
Ac-Lys-(Ac-Lys-Lys)Lys-SEQ ID NO: 1:
Ac-Lys-(Ac-Lys-Lys)Lys-His-Gly-Glu-Gly-Thr-Phe-
Thr-Ser-Asp-Leu-Ser-Lys-Gln-Met-Glu-Glu-Glu-Ala-
Val-Arg-Leu-Phe-Ile-Glu-Trp-Leu-Lys-Asn-Gly-Gly-
Pro-Ser-Ser-Gly-Ala-Pro-Pro-Ser
SEQ ID NO: 1-Lys-(Ac-Lys-Lys)Lys:
His-Gly-Glu-Gly-Thr-Phe-Thr-Ser-Asp-Leu-Ser-Lys-
Gln-Met-Glu-Glu-Glu-Ala-Val-Arg-Leu-Phe-Ile-Glu-
Trp-Leu-Lys-Asn-Gly-Gly-Pro-Ser-Ser-Gly-Ala-Pro-
Pro-Ser-Lys-(Ac-Lys-Lys)Lys
Ac-(Ac-Lys)Lys-(Ac-Lys-)Lys-SEQ ID NO: 1:
Ac-(Ac-Lys)Lys-(Ac-Lys-)Lys-His-Gly-Glu-Gly-Thr-
Phe-Thr-Ser-Asp-Leu-Ser-Lys-Gln-Met-Glu-Glu-Glu-
Ala-Val-Arg-Leu-Phe-Ile-Glu-Trp-Leu-Lys-Asn-Gly-
Gly-Pro-Ser-Ser-Gly-Ala-Pro-Pro-Ser
SEQ ID NO: 1-(Ac-Lys)Lys-(Ac-Lys-)Lys:
His-Gly-Glu-Gly-Thr-Phe-Thr-Ser-Asp-Leu-Ser-Lys-
Gln-Met-Glu-Glu-Glu-Ala-Val-Arg-Leu-Phe-Ile-Glu-
Trp-Leu-Lys-Asn-Gly-Gly-Pro-Ser-Ser-Gly-Ala-Pro-
Pro-Ser-(Ac-Lys)Lys-(Ac-Lys-)Lys
Ac-Lys-Lys-(Ac-Lys)Lys-SEQ ID NO: 1:
Ac-Lys-Lys-(Ac-Lys)Lys-His-Gly-Glu-Gly-Thr-Phe-
Thr-Ser-Asp-Leu-Ser-Lys-Gln-Met-Glu-Glu-Glu-Ala-
Val-Arg-Leu-Phe-Ile-Glu-Trp-Leu-Lys-Asn-Gly-Gly-
Pro-Ser-Ser-Gly-Ala-Pro-Pro-Ser
SEQ ID NO: 1-Lys-Lys-(Ac-Lys)Lys:
His-Gly-Glu-Gly-Thr-Phe-Thr-Ser-Asp-Leu-Ser-Lys-
Gln-Met-Glu-Glu-Glu-Ala-Val-Arg-Leu-Phe-Ile-Glu-
Trp-Leu-Lys-Asn-Gly-Gly-Pro-Ser-Ser-Gly-Ala-Pro-
Pro-Ser-Lys-Lys-(Ac-Lys)Lys
Ac-Lys-(Ac-Lys)Lys-Lys-SEQ ID NO: 1:
Ac-Lys-(Ac-Lys)Lys-Lys-His-Gly-Glu-Gly-Thr-Phe-
Thr-Ser-Asp-Leu-Ser-Lys-Gln-Met-Glu-Glu-Glu-Ala-
Val-Arg-Leu-Phe-Ile-Glu-Trp-Leu-Lys-Asn-Gly-Gly-
Pro-Ser-Ser-Gly-Ala-Pro-Pro-Ser
SEQ ID NO: 1-Lys-(Ac-Lys)Lys-Lys:
His-Gly-Glu-Gly-Thr-Phe-Thr-Ser-Asp-Leu-Ser-Lys-
Gln-Met-Glu-Glu-Glu-Ala-Val-Arg-Leu-Phe-Ile-Glu-
Trp-Leu-Lys-Asn-Gly-Gly-Pro-Ser-Ser-Gly-Ala-Pro-
Pro-Ser-Lys-(Ac-Lys)Lys-Lys
Ac-(Ac-Lys)Lys-Lys-Lys-SEQ ID NO: 1:
Ac-(Ac-Lys)Lys-Lys-Lys-His-Gly-Glu-Gly-Thr-Phe-
Thr-Ser-Asp-Leu-Ser-Lys-Gln-Met-Glu-Glu-Glu-Ala-
Val-Arg-Leu-Phe-Ile-Glu-Trp-Leu-Lys-Asn-Gly-Gly-
Pro-Ser-Ser-Gly-Ala-Pro-Pro-Ser
SEQ ID NO: 1-(Ac-Lys)Lys-Lys-Lys:
His-Gly-Glu-Gly-Thr-Phe-Thr-Ser-Asp-Leu-Ser-Lys-
Gln-Met-Glu-Glu-Glu-Ala-Val-Arg-Leu-Phe-Ile-Glu-
Trp-Leu-Lys-Asn-Gly-Gly-Pro-Ser-Ser-Gly-Ala-Pro-
Pro-Ser-(Ac-Lys)Lys-Lys-Lys
Ac-(Ac-Lys-Gly)Lys-Lys-SEQ ID NO: 1:
Ac-(Ac-Lys-Gly)Lys-Lys-His-Gly-Glu-Gly-Thr-Phe-
Thr-Ser-Asp-Leu-Ser-Lys-Gln-Met-Glu-Glu-Glu-Ala-
Val-Arg-Leu-Phe-Ile-Glu-Trp-Leu-Lys-Asn-Gly-Gly-
Pro-Ser-Ser-Gly-Ala-Pro-Pro-Ser
SEQ ID NO: 1-(Ac-Lys-Gly)Lys-Lys:
His-Gly-Glu-Gly-Thr-Phe-Thr-Ser-Asp-Leu-Ser-Lys-
Gln-Met-Glu-Glu-Glu-Ala-Val-Arg-Leu-Phe-Ile-Glu-
Trp-Leu-Lys-Asn-Gly-Gly-Pro-Ser-Ser-Gly-Ala-Pro-
Pro-Ser-(Ac-Lys-Gly)Lys-Lys
Ac-(Ac-Lys-Gly)Lys-Lys-SEQ ID NO: 1:
Ac-(Ac-Lys-Gly)Lys-Lys-His-Gly-Glu-Gly-Thr-Phe-
Thr-Ser-Asp-Leu-Ser-Lys-Gln-Met-Glu-Glu-Glu-Ala-
Val-Arg-Leu-Phe-Ile-Glu-Trp-Leu-Lys-Asn-Gly-Gly-
Pro-Ser-Ser-Gly-Ala-Pro-Pro-Ser
SEQ ID NO: 1-(Ac-Lys-Gly)Lys-Lys:
His-Gly-Glu-Gly-Thr-Phe-Thr-Ser-Asp-Leu-Ser-Lys-
Gln-Met-Glu-Glu-Glu-Ala-Val-Arg-Leu-Phe-Ile-Glu-
Trp-Leu-Lys-Asn-Gly-Gly-Pro-Ser-Ser-Gly-Ala-Pro-
Pro-Ser-(Ac-Lys-Gly)Lys-Lys
SEQ ID NO: 1-[(Ac-Lys-Lys-Lys)Lys-]_{Lys12}:
His-Gly-Glu-Gly-Thr-Phe-Thr-Ser-Asp-Leu-Ser-[Ac-
Lys-Lys-Lys)Lys]Lys-Gln-Met-Glu-Glu-Glu-Ala-Val-
Arg-Leu-Phe-Ile-Glu-Trp-Leu-Lys-Asn-Gly-Gly-Pro-
Ser-Ser-Gly-Ala-Pro-Pro-Ser
SEQ ID NO: 1-[(Ac-Lys-Lys-Lys)Lys-]_{Lys27}:
His-Gly-Glu-Gly-Thr-Phe-Thr-Ser-Asp-Leu-Ser-Lys-
Gln-Met-Glu-Glu-Glu-Ala-Val-Arg-Leu-Phe-Ile-Glu-
Trp-Leu-[Ac-Lys-Lys-Lys)Lys]-Lys-Asn-Gly-Gly-
Pro-Ser-Ser-Gly-Ala-Pro-Pro-Ser

The N-terminus may be acetylated (Ac) or H— and the C-terminus may be amidated (NH₂).

### EXAMPLES

### Example 1—Synthesis of BAP-Modified Exendin-4 Peptide Analogues

BAP modified peptides are synthesized using standard Fmoc chemistry using 1-[Bis(dimethylamino)methylene]-1H-1,2,3-triazolo[4,5-b]pyridinium 3-oxid hexafluorophosphate (HATU) or 2-(6-Chloro-1H-benzotriazole-1-yl)-1,1,3,3-tetramethylaminium hexafluorophosphate (HCTU) as the coupling reagents with Hunig's Base (N,N-diisopropylethylamine, DIPEA). For the lysine branching as described in more detail below, combination of orthogonally protected lysines is used including Fmoc-Lys(MTT)-OH, FmocLys(ivDde)-OH, and Fmoc-Lys(Boc)-OH.

Peptides are cleaved with standard cleavage cocktails including trifluoroacetic acid, triisoproproylsilane, and water and precipitated with ice-cold ether. All crude peptides are purified by reversed-phase chromatography on columns with C-18 functionality and using gradients of acetonitrile, deionized water, and trifluoroacetic acid as running buffers. Purity is determined by high-pressure liquid chromatography and mass (MS) and sequence (tandem MS) information was obtained using a nanospray mass spectrometer.

BAP Attached in the C-Terminus of the Sequence

Branching on the C-terminal lysine (METHOD 1): N-α-Fmoc-N-ε-4-methyltrityl-L-lysine or N-α-Fmoc-N-ε-1-(4,4-dimethyl-2,6-dioxocyclohex-1-ylidene)-3-methylbutyl-L-lysine is added to Rink amide resin after piperidine deprotection. The remaining sequence of the target peptide is added and the full length sequence is acetylated with acetic anhydride. The lysine side chain protecting is then removed using 1% trifluoroacetic acid in dichloromethane (Mtt) or hydroxylamine hydrochloride/imidazole in NMP (ivDde). Additional Nα-Fmoc-Nε-Boc-L-lysine is then added to the side chain and acetylated when desired.

Branching on other than the C-terminal lysine: analogously to attaching BAP to alkylamines in the sequence between the N- and C-termini (METHOD 2).

BAP Covalently Linked to Lysines in the Sequence Between the N- and C-Termini

METHOD 2: N-α-Fmoc-N-ε-4-methyltrityl-L-lysine or N-α-Fmoc-N-ε-1-(4,4-dimethyl-2,6-dioxocyclohex-1-ylidene)-3-methylbutyl-L-lysine is added to the peptide sequence, lysine side chain protecting group is removed after finalizing the sequence and optionally N-terminal acetylation. Appropriate lysine analogues such as Fmoc-Lys(MTT)-OH, Fmoc-Lys(ivDde)-OH and Fmoc-Lys(Boc)-OH is sequentially added and selectively deprotected, before acetylation to ensure appropriate side chain and acetyl addition.

BAP is added to other amino alkyl residues than lysine by analogously using Fmoc/4-methyltrityl protected amino alkyl amino acids.

BAP Attached in the N-Terminus of the Sequence

Branching on the N-terminal lysine (METHOD 3): N-α-Fmoc-N-ε-4-methyltrityl-L-lysine is added to N-terminal of the sequence, Fmoc is removed, the sequence acetylated at the N-terminus, and the metyltrityl group is removed. Additional Nα-Fmoc-Nε-Boc-L-lysine is then added to the side chain and acetylated when desired.

Branching on other than the N-terminal lysine: analogously to attaching BAP to lysines in the sequence between the N- and C-termini (METHOD 2).

### Example 2: Pharmacological Characterization of BAP-Modified Exendin-4 Peptide Analogues

The potency and efficacy of the exendin-4 analogues can be determined using different pharmacological procedures. The present invention is further illustrated with reference to the following examples, which are not intended to be limiting in any way to the scope of the invention as claimed.

CHO-K1 cells expressing the human GLP-1 receptor grown in media without antibiotic were detached by gentle flushing with PBS-EDTA (5 mM EDTA), recovered by centrifugation and resuspended in assay buffer (KRH: 5 mM KCl, 1.25 mM MgSO4, 124 mM NaCl, 25 mM HEPES, 13.3 mM Glucose, 1.25 mM KH2PO4, 1.45 mM CaCl2), 0.5 g/I BSA).

12 μl of cells were mixed with 12 μl of the test compound (solubilized in PBS/0.5% BSA and finally diluted from a stock solution of 1 mM) at increasing concentrations in 96 wells plates and then incubated 30 min at room temperature. cAMP production was determined after addition of a lysis buffer and 1 hour incubation, by use of competitive immunoassay using cryptate-labeled anti-cAMP and d2-labeled cAMP (HTRF kit from CisBio) with Delta F percentage values calculated according to the manufacturer specification. Dose response curves were performed in parallel with test compounds, and reference compounds.

The HTRF technology is a titration assay based on a competition between labeled cAMP (exogenous) and cAMP produced by the cell after activation of the receptor. The dynamic range of the assay was 3-4 fold meaning that the linear range (which enables conversion from raw data to nM of cAMP) is within that range. The window between top and bottom of the curve is higher (around 100) which means that converting into nM of cAMP, the assay window of cAMP goes from 1 nM (basal) to around 30 nM (Emax). All experiments were conducted in the presence of the non-selective phosphodiesterase inhibitor IBMX (1 mM in final concentration).

The test compounds were tested in a concentration range from 10⁻¹⁴ to 10⁻⁷ M Data is presented as mean values. The EC50 (ie the concentration induced 50% of max response) was determined by best fit analyses after logarithmic transformation using the graph pad software (version 6.0)

Reference Compound/Control Peptide 1: GLP-1 (7-36):

(SEQ ID NO: 6)
H-His-Ala-Glu-Gly-Thr-Phe-Thr-Ser-Asp-Val-Ser-
Ser-Tyr-Leu-Glu-Gly-Gln-Ala-Ala-Lys-Glu-Phe-Ile-
Ala-Trp-Leu-Val-Lys-Gly-Arg-Gly-OH

Reference Compound/Control Peptide 2:

SEQ ID NO: 1-(Lys)₆-NH2;
or
(SEQ ID NO: 5)
des-Pro38-Exendin-4-SIP having the sequence His-
Gly-Glu-Gly-Thr-Phe-Thr-Ser-Asp-Leu-Ser-Lys-Gln-
Met-Glu-Glu-Glu-Ala-Val-Arg-Leu-Phe-Ile-Glu-Trp-
Leu-Lys-Asn-Gly-Gly-Pro-Ser-Ser-Gly-Ala-Pro-Pro-
Ser-(Lys)₆-NH₂

Test Compound—Analogue 1:

SEQ ID NO: 1-(Ac-Lys-Lys)Lys-NH2;
or
(SEQ ID NO: 1-(Ac-Lys-Lys)Lys-NH2)
des-Pro³⁸-Exendin-4-BAP having the sequence His-
Gly-Glu-Gly-Thr-Phe-Thr-Ser-Asp-Leu-Ser-Lys-Gln-
Met-Glu-Glu-Glu-Ala-Val-Arg-Leu-Phe-Ile-Glu-Trp-
Leu-Lys-Asn-Gly-Gly-Pro-Ser-Ser-Gly-Ala-Pro-Pro-
Ser-(Ac-Lys-Lys)Lys-NH₂

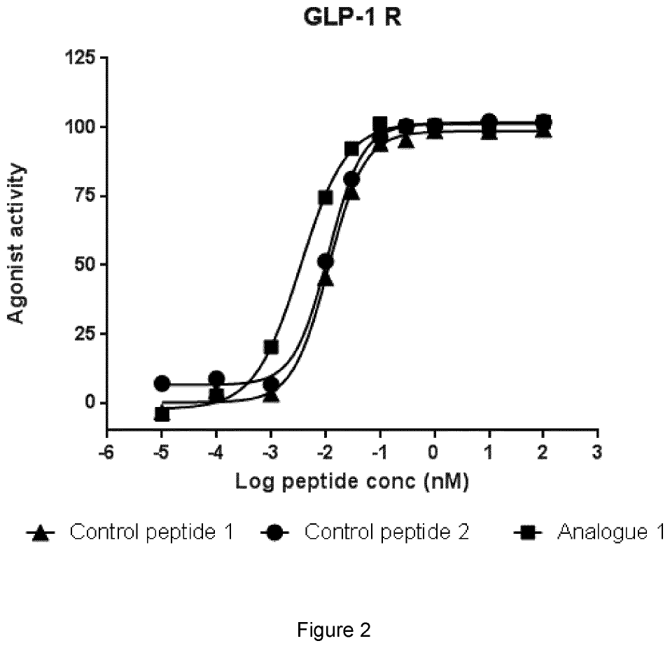

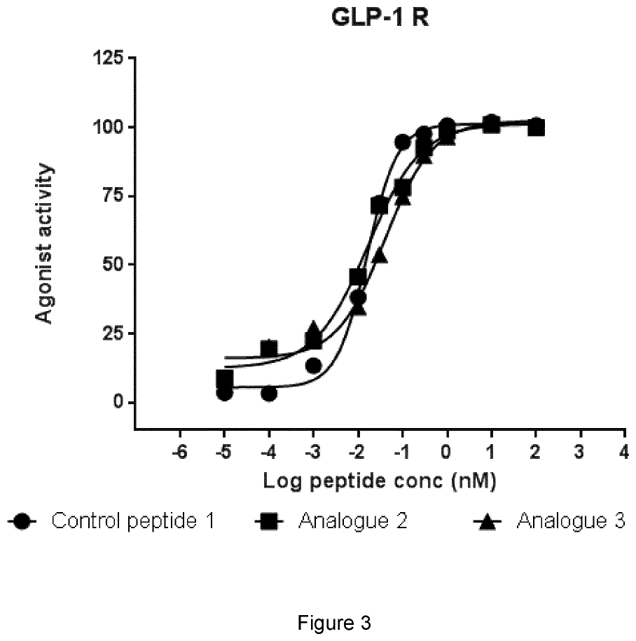

Results (See Also FIG. 2):

| | Control peptide 1: | Control peptide 2: | Analogue 1: |
|---|---|---|---|
| EC50 (nM) | 0.011 | 0.011 | 0.003 |

Surprisingly, the exendin-4 peptide (analogue 1) in addition to showing full agonist activity when compared to both control peptide 1 and 2, also proved to be a very potent agonist to the GLP-1 receptor with an EC₅₀ almost one decade lower that what was found for the two control peptides. Especially it is surprising that BAP modification with C-terminal -(Ac-Lys-Lys)Lys-NH₂ of SEQ ID NO:1 was associated with increased potency when compared to C-terminal modification of SEQ ID NO:1 with (Lys)₆-NH₂.

### Example 3

The data shown in FIG. 3 is included and detailed in PCT/IB2015/000553 (WO/2015/162485);

| | Control peptide 1: | Analogue 2: | Analogue 3: |
|---|---|---|---|
| EC50 (nM) | 0.02 | 0.02 | 0.04 |

Control peptide 1:
(SEQ ID NO: 6)
GLP-1 (7-36) having sequence His-Ala-Glu-Gly-
Thr-Phe-Thr-Ser-Asp-Val-Ser-Ser-Tyr-Leu-Glu-Gly-
Gln-Ala-Ala-Lys-Glu-Phe-Ile-Ala-Trp-Leu-Val-Lys-
Gly-Arg-Gly;
Analogue 2:
having the sequence Ac-His-Ala-Glu-
Gly-Thr-Phe-Thr-Ser-Asp-Val-Ser-Ser-Tyr-Leu-Glu-
Gly-Gln-Ala-Ala-Lys-Glu-Phe-Ile-Ala-Trp-Leu-Val-
Lys-Gly-Arg-Gly-(Lys-Lys-Ac)Lys-NH₂;
and
Analogue 3:
having the sequence Ac-His-Gly-Glu-Gly-Thr-Phe-
Thr-Ser-Asp-Val-Ser-Ser-Tyr-Leu-Glu-Gly-Gln-Ala-
Ala-Lys-Glu-Phe-Ile-Ala-Trp-Leu-Val-Lys-Gly-Arg-
Gly-(Lys-Lys-Ac)Lys-NH₂.

These results demonstrate that not all peptides are made more potent or even retains their potency by the presence of a C terminal BAP.

1. An exendin-4 peptide analogue comprising an exendin-4 peptide and one or more branched amino acid probes,
wherein said branched amino acid probe comprises a first amino alkyl amino acid residue,
said first amino alkyl amino acid residue optionally being covalently linked to a second amino alkyl amino acid residue, or to a second and a third amino alkyl amino acid residue, to form a linear chain of 2 or 3 amino alkyl amino acid residues,
wherein the side chain of one or more of said first, second and/or third amino alkyl amino acid residues are each modified by attaching to the side chain amino group a molecule independently selected from the group consisting of AAA_{q}-AAA; (aa₃)ₚ-AAA_{q}; AAA_{q}-(aa₃)ₚ; [(aa₃)-AAA]ₚ and [AAA-(aa₃)]ₚ;
wherein q is a number selected from 0, 1, 2 and 3; p is a number selected from 1, 2 and 3; AAA is an amino alkyl amino acid residue; and (aa₃) is an amino acid residue independently selected from Arg, His, Gly and Ala,
wherein said first amino alkyl amino acid residue is covalently linked to the N-terminus of said exendin-4 peptide analogue, covalently linked to the C-terminus of said exendin-4 peptide analogue, and/or covalently linked to the side chain amino group of an amino alkyl amino acid residue within said exendin-4 peptide analogue,
with the proviso that said branched amino acid probe consists of 2 to 9 amino acid residues, and
wherein said exendin-4 peptide is selected from the group consisting of des-Pro³⁸-exendin-4(1-39) (SEQ ID NO:1), des-Ser³⁹-exendin-4(1-39) (SEQ ID NO:2) and exendin-4(1-39) (SEQ ID NO:3), or a functional variant thereof.

2. The exendin-4 peptide analogue according to claim 1, wherein said amino alkyl amino acid residue is an amino acid with a side chain comprising an amino alkyl group (—CₙH₂ₙNH₂); such as a side chain amino alkyl group selected from the group consisting of methylamine (—CH₂NH₂), ethylamine (—C₂H₄NH₂), propylamine (—C₃H₆NH₂), n-butylamine (—C₄H₈NH₂), pentylamine (—C₅H₁₀NH₂), n-hexylamine (—C₆H₁₂NH₂), heptylamine (—C₇H₁₄NH₂), octylamine (—C₈H₁₆NH₂), nonylamine (—C₉H₁₈NH₂), decylamine (—C₁₀H₂₀NH₂), undecylamine (—C₁₁H₂₂NH₂) and dodecylamine (—C₁₂H₂₄NH₂).

3. The exendin-4 peptide analogue according to the preceding claims, wherein the side chain amino group of said amino alkyl amino acid residue is selected from the group consisting of the β-amino group (methylamine); the γ-amino group (ethylamine); the δ-amino group (propylamine), the ε-amino group (n-butylamine); the ζ-amino group (pentylamine); the η-amino group (n-hexylamine); the θ-amino group (heptylamine); the ι-amino group (octylamine); the κ-amino group (nonylamine); the λ-amino group (decylamine); the μ-amino group (undecylamine); and the v-amino group (dodecylamine).

4. The exendin-4 peptide analogue according to the preceding claims, wherein said branched amino acid probe comprises
a. a first amino alkyl amino acid residue,
b. a first and a second amino alkyl amino acid residue, or
c. a first, a second and a third amino alkyl amino acid residue.

5. The exendin-4 peptide analogue according to the preceding claims, wherein said branched amino acid probe comprises
a. a first amino alkyl amino acid residue, wherein the N-terminus of said first amino alkyl amino acid residue is acetylated,
b. a first and a second amino alkyl amino acid residue, wherein the N-terminus of said second amino alkyl amino acid residue is acetylated, or
c. a first, a second and a third amino alkyl amino acid residue, wherein the N-terminus of said third amino alkyl amino acid residue is acetylated.

6. The exendin-4 peptide analogue according to the preceding claims, wherein said branched amino acid probe comprises
a. a first amino alkyl amino acid residue, wherein the C-terminus of said first amino alkyl amino acid residue is a carboxylic acid, an aldehyde, an ester, or an amide, such as a primary amide (CONH₂),
b. a first and a second amino alkyl amino acid residue, wherein the C-terminus of said second amino alkyl amino acid residue is a carboxylic acid, an aldehyde, an ester, or an amide, such as a primary amide (CONH₂), or
c. a first, a second and a third amino alkyl amino acid residue, wherein the C-terminus of said third amino alkyl amino acid residue is a carboxylic acid, an aldehyde, an ester, or an amide, such as a primary amide (CONH₂).

7. The exendin-4 peptide analogue according to the preceding claims, wherein the N-terminal amino acid residue of the molecule is acetylated at the alpha amino group.

8. The exendin-4 peptide analogue according to the preceding claims, wherein the branched amino acid probe comprises a first amino alkyl amino acid residue, said first amino alkyl amino acid residue being optionally N-terminal acetylated or C-terminal amidated,
wherein the side chain amino group of said first amino alkyl amino acid residue is modified by attaching a molecule independently selected from the group consisting of AAA_{q}-AAA; (aa₃)ₚ-AAA_{q}; AAA_{q}-(aa₃)ₚ; [(aa₃)-AAA]ₚ and [AAA-(aa₃)]ₚ;
wherein q is a number selected from 0, 1, 2 and 3; p is a number selected from 1, 2 and 3; AAA is an amino alkyl amino acid residue; (aa₃) is an amino acid residue independently selected from Arg, His, Gly and Ala, and the N-terminal AAA or (aa)₃ of the molecule is optionally acetylated.

9. The exendin-4 peptide analogue according to the preceding claims, wherein the branched amino acid probe comprises a first amino alkyl amino acid residue covalently linked to a second amino alkyl amino acid residue, to form a linear chain of 2 amino alkyl amino acid residues,
said second amino alkyl amino acid residue being optionally N-terminal acetylated or C-terminal amidated,
wherein the side chain amino group of said first and/or said second amino alkyl amino acid residue is modified by attaching a molecule independently selected from the group consisting of AAA_{q}-AAA; (aa₃)ₚ-AAA_{q}; AAA_{q}-(aa₃)ₚ; [(aa₃)-AAA]ₚ and [AAA-(aa₃)]ₚ; wherein q is a number selected from 0, 1, 2 and 3; p is a number selected from 1, 2 and 3; AAA is an amino alkyl amino acid residue; (aa₃) is an amino acid residue independently selected from Arg, His, Gly and Ala, and the N-terminal AAA or (aa)₃ of the molecule is optionally acetylated.

10. The exendin-4 peptide analogue according to the preceding claims, wherein the branched amino acid probe comprises a first and a second amino alkyl amino acid residue, wherein the side chain of said first amino alkyl amino acid residue is modified by attaching a molecule to the side chain amino group.

11. The exendin-4 peptide analogue according to the preceding claims, wherein the branched amino acid probe comprises a first and a second amino alkyl amino acid residue, wherein the side chain of said second amino alkyl amino acid residue is modified by attaching a molecule to the side chain amino group.

12. The exendin-4 peptide analogue according to the preceding claims, wherein the branched amino acid probe comprises a first amino alkyl amino acid residue covalently linked to a second and a third amino alkyl amino acid residue to form a linear chain of 3 amino alkyl amino acid residues,
said third amino alkyl amino acid residue being optionally N-terminal acetylated or C-terminal amidated,
wherein the side chain amino group of said first, second and/or third amino alkyl amino acid residues is modified by attaching a molecule independently selected from the group consisting of AAA_{q}-AAA; (aa₃)ₚ-AAA_{q}; AAA_{q}-(aa₃)ₚ; [(aa₃)-AAA]ₚ and [AAA-(aa₃)]ₚ; wherein q is a number selected from 0, 1, 2 and 3; p is a number selected from 1, 2 and 3; AAA is an amino alkyl amino acid residue; (aa₃) is an amino acid residue independently selected from Arg, His, Gly and Ala, and the N-terminal AAA or (aa)₃ of the molecule is optionally acetylated.

13. The exendin-4 peptide analogue according to the preceding claims, wherein the branched amino acid probe comprises a first, a second and a third amino alkyl amino acid residue, wherein
a. the side chain of said first amino alkyl amino acid residue is modified by attaching a molecule to the side chain amino group,
b. the side chain of said second amino alkyl amino acid residue is modified by attaching a molecule to the side chain amino group,
c. the side chain of said third amino alkyl amino acid residue is modified by attaching a molecule to the side chain amino group,
d. the side chain of said first and second amino alkyl amino acid residue is modified by attaching a molecule to the side chain amino group,
e. the side chain of said first and third amino alkyl amino acid residue is modified by attaching a molecule to the side chain amino group,
f. the side chain of said second and third amino alkyl amino acid residue is modified by attaching a molecule to the side chain amino group, or
g. the side chain of said first, second and third amino alkyl amino acid residue is modified by attaching a molecule to the side chain amino group.

14. The exendin-4 peptide analogue according to the preceding claims, wherein the amino alkyl amino acid residues of the branched amino acid probe are individually selected from the group consisting of lysine and ornithine.

15. The exendin-4 peptide analogue according to the preceding claims, wherein each of the first, second and/or third amino alkyl amino acids of the branched amino acid probe are individually selected from the group consisting of lysine and ornithine.

16. The exendin-4 peptide analogue according to the preceding claims, wherein each AAA of the molecules AAA_{q}-AAA; (aa₃)ₚ-AAA_{q}; AAA_{q}-(aa₃)ₚ; [(aa₃)-AAA]ₚ and [AAA-(aa₃)]ₚ are individually selected from the group consisting of lysine and ornithine.

17. The exendin-4 peptide analogue according to the preceding claims, wherein said amino acid residues of said branched amino acid probe each are the same or different.

18. The exendin-4 peptide analogue according to the preceding claims, wherein said side chain amino group is individually selected from the δ-amino group (ornithine) and the ε-amino group (lysine).

19. The exendin-4 peptide analogue according to the preceding claims, wherein the molecule to be covalently linked to said side chain amino group is independently selected from the group consisting of Lys_{q}-Lys; (aa₃)ₚ-Lys_{q}; Lys_{q}-(aa₃)ₚ; [(aa₃)-Lys]ₚ; [Lys-(aa₃)]ₚ; Orn_{q}-Orn; (aa₃)ₚ-Orn_{q}; Orn_{q}-(aa₃)ₚ; [(aa₃)-Orn]ₚ and [Orn-(aa₃)]ₚ; Ornₚ-Lysₚ; Lysₚ-Ornₚ; [Orn-Lys]ₚ and [Lys-Orn]ₚ, wherein q is a number selected from 0, 1, 2 and 3; p is a number selected from 1, 2 and 3; (aa₃) is an amino acid residue independently selected from Arg, His, Gly and Ala; and the N-terminal Lys, Orn or (aa)₃ amino acid residue is optionally acetylated at the alpha amino group.

20. The exendin-4 peptide analogue according to the preceding claims, wherein the molecule to be covalently linked to said side chain amino group is independently selected from the group consisting of Lys_{q}-Lys; Orn_{q}-Orn; Ornₚ-Lysₚ; Lysₚ-Ornₚ; [Orn-Lys]ₚ and [Lys-Orn]ₚ, wherein q is a number selected from 0, 1, 2 and 3; p is a number selected from 1, 2 and 3; and the N-terminal Lys or Orn amino acid residue is optionally acetylated at the alpha amino group.

21. The exendin-4 peptide analogue according to the preceding claims, wherein the molecule to be covalently linked to said side chain amino group is independently selected from the group consisting of Lys_{q}-Lys; (aa₃)ₚ-Lys_{q}; Lys_{q}(aa₃)ₚ; [(aa₃)-Lys]ₚ; and [Lys-(aa₃)]ₚ; and the N-terminal Lys or (aa₃) residue is optionally acetylated at the alpha amino group.

22. The exendin-4 peptide analogue according to the preceding claims, wherein the amino alkyl amino acid residues of the branched amino acid probe are lysine residues.

23. The exendin-4 peptide analogue according to the preceding claims, wherein the molecule to be covalently linked to said side chain amino group is Lys_{q}-Lys; wherein q is a number selected from 0, 1, 2 and 3 and said the N-terminal Lys residue is optionally acetylated at the alpha amino group.

24. The exendin-4 peptide analogue according to the preceding claims, wherein the molecule to be covalently linked to said side chain amino group is independently selected from the group consisting of
Ac-AAA_{q}-AAA; Ac-(aa₃)ₚ-AAA_{q}; Ac-AAA_{q}-(aa₃)ₚ; Ac-[(aa₃)-AAA]ₚ; Ac-[AAA-(aa₃)]ₚ,
Ac-Lys_{q}-Lys; Ac-(aa₃)ₚ-Lys_{q}; Ac-Lys_{q}-(aa₃)ₚ; Ac-[(aa₃)-Lys]ₚ; Ac-[Lys-(aa₃)]ₚ; Ac-Orn_{q}-Orn; Ac-(aa₃)ₚ-Orn_{q}; Ac-Orn_{q}-(aa₃)ₚ; Ac-[(aa₃)-Orn]ₚ; Ac-[Orn-(aa₃)]ₚ; Ac-Ornₚ-Lysₚ; Ac-Lysₚ-Ornₚ; Ac-[Orn-Lys]ₚ and Ac-[Lys-Orn]ₚ,
wherein q is a number selected from 0, 1, 2 and 3; p is a number selected from 1, 2 and 3; and (aa₃) is an amino acid residue independently selected from Arg, His, Gly and Ala.

25. The exendin-4 peptide analogue according to the preceding claims, wherein the branched amino acid probe consist of 2 to 3 amino acid residues, such as 3 to 4 amino acid residues, for example 4 to 5 amino acid residues, such as 5 to 6 amino acid residues, for example 6 to 7 amino acid residues, such as 7 to 8 amino acid residues, for example 8 to 9 amino acid residues.

26. The exendin-4 peptide analogue according to the preceding claims, wherein the branched amino acid probe consist of 2 amino acid residues, such as 3 amino acid residues, for example 4 amino acid residues, such as 5 amino acid residues, for example 6 amino acid residues, such as 7 amino acid residues, for example 8 amino acid residues, such as 9 amino acid residues.

27. The exendin-4 peptide analogue according to the preceding claims, wherein the molecule to be covalently linked to the side chain amino group(s) of one or more of the first, second and/or third amino alkyl amino acid residues is selected from the group consisting of AAA, Ac-AAA, AAA-AAA, Ac-AAA-AAA, AAA-AAA-AAA, Ac-AAA-AAA-AAA, AAA-AAA-AAA-AAA, Ac-AAA-AAA-AAA-AAA, AAA-Gly-AAA, Ac-AAA-Gly-AAA, AAA-AAA-Gly, Ac-AAA-AAA-Gly, AAA-Gly, Ac-AAA-Gly, AAA-Ala-AAA, Ac-AAA-Ala-AAA, AAA-AAA-Ala, Ac-AAA-AAA-Ala, AAA-Ala, Ac-AAA-Ala, AAA-His-AAA, Ac-AAA-His-AAA, AAA-AAA-His, Ac-AAA-AAA-His, AAA-His, Ac-AAA-His, AAA-Arg-AAA, Ac-AAA-Arg-AAA, AAA-AAA-Arg, Ac-AAA-AAA-Arg, AAA-Arg and Ac-AAA-Arg; wherein AAA is an amino alkyl amino acid residue.

28. The exendin-4 peptide analogue according to the preceding claims, wherein the molecule to be covalently linked to the side chain amino group(s) of one or more of the first, second and/or third amino alkyl amino acid residues is selected from the group consisting of Lys, Ac-Lys, Lys-Lys, Ac-Lys-Lys, Lys-Lys-Lys, Ac-Lys-Lys-Lys, Lys-Lys-Lys-Lys, Ac-Lys-Lys-Lys-Lys, Lys-Gly-Lys, Ac-Lys-Gly-Lys, Lys-Lys-Gly, Ac-Lys-Lys-Gly, Lys-Gly, Ac-Lys-Gly, Lys-Ala-Lys, Ac-Lys-Ala-Lys, Lys-Lys-Ala, Ac-Lys-Lys-Ala, Lys-Ala, Ac-Lys-Ala, Lys-His-Lys, Ac-Lys-His-Lys, Lys-Lys-His, Ac-Lys-Lys-His, Lys-His, Ac-Lys-His, Lys-Arg-Lys, Ac-Lys-Arg-Lys, Lys-Lys-Arg, Ac-Lys-Lys-Arg, Lys-Arg and Ac-Lys-Arg.

29. The exendin-4 peptide analogue according to the preceding claims, wherein said branched amino acid probe is selected from the group consisting of
a. (AAA)AAA₁-, (AAA-AAA)AAA₁-, (AAA-AAA-AAA)AAA₁-, (AAA-AAA-AAA-AAA)AAA₁, (AAA-Gly-AAA)AAA₁-, (AAA-AAA-Gly)AAA₁-, (AAA-Gly)AAA₁-, (AAA-Ala-AAA)AAA₁-, (AAA-AAA-Ala)AAA₁-, (AAA-Ala)AAA₁-, (AAA-His-AAA)AAA₁-, (AAA-AAA-His)AAA₁-, (AAA-His)AAA₁-, (AAA-Arg-AAA)AAA₁-, (AAA-AAA-Arg)AAA₁-, and (AAA-Arg)AAA₁-, wherein said first amino alkyl amino acid reside (AAA₁-) is optionally N-terminally acetylated or C-terminally amidated;
b. (Lys)Lys₁-, (Lys-Lys)Lys₁-, (Lys-Lys-Lys)Lys₁-, (Lys-Lys-Lys-Lys)Lys₁-, (Lys-Gly-Lys)Lys₁-, (Lys-Lys-Gly)Lys₁-, (Lys-Gly)Lys₁-, (Lys-Ala-Lys)Lys₁-, (Lys-Lys-Ala)Lys₁-, (Lys-Ala)Lys₁-, (Lys-His-Lys)Lys₁-, (LysLys-His)Lys₁-, (Lys-His)Lys₁-, (Lys-Arg-Lys)Lys₁-, (Lys-Lys-Arg)Lys₁-, and (Lys-Arg)Lys₁-, wherein said first lysine reside (Lys₁-) is optionally N-terminally acetylated or C-terminally amidated;
c. Ac-(Ac-Lys)Lys₁-, Ac-(Ac-Lys-Lys)Lys₁-, Ac-(Ac-Lys-Lys-Lys)Lys₁-, Ac-(Ac-Lys-Lys-Lys-Lys)Lys₁-, Ac-(Ac-Lys-Gly-Lys)Lys₁-, Ac-(Ac-Lys-Lys-Gly)Lys₁-, Ac-(Ac-Lys-Gly)Lys₁-, Ac-(Ac-Lys-Ala-Lys)Lys₁-, Ac-(Ac-LysLys-Ala)Lys₁-, Ac-(Ac-Lys-Ala)Lys₁-, Ac-(Ac-Lys-His-Lys)Lys₁-, Ac-(Ac-Lys-Lys-His)Lys₁-, Ac-(Ac-Lys-His)Lys₁-, Ac-(Ac-Lys-Arg-Lys)Lys₁-, Ac-(Ac-Lys-Lys-Arg)Lys₁-, and Ac-(Ac-Lys-Arg)Lys₁-, or
d. (Ac-Lys)Lys₁-NH₂, (Ac-Lys-Lys)Lys₁-NH₂, (Ac-Lys-Lys-Lys)Lys₁-NH₂, (Ac-Lys-Lys-Lys-Lys)Lys₁-NH₂, (Ac-Lys-Gly-Lys)Lys₁-NH₂, (Ac-Lys-Lys-Gly)Lys₁-NH₂, (Ac-Lys-Gly)Lys₁-NH₂, (Ac-Lys-Ala-Lys)Lys₁-NH₂, (Ac-Lys-Lys-Ala)Lys₁-NH₂, (Ac-Lys-Ala)Lys₁-NH₂, (Ac-Lys-His-Lys)Lys₁-NH₂, (Ac-Lys-Lys-His)Lys₁-NH₂, (Ac-Lys-His)Lys₁-NH₂, (Ac-Lys-Arg-Lys)Lys₁-NH₂, (Ac-Lys-Lys-Arg)Lys₁-NH₂, and (Ac-Lys-Arg)Lys₁-NH₂.

30. The exendin-4 peptide analogue according to the preceding claims, wherein the branched amino acid probe is selected from the group consisting of
Ac-(Ac-Lys)Lys-Lys-, (Ac-Lys)Lys-Lys-, Ac-(Lys)Lys-Lys-, (Lys)Lys-Lys-, (Ac-Lys)Lys-Lys-NH₂, (Lys)Lys-Lys-NH₂;
Ac-Lys-(Ac-Lys)Lys-, Lys-(Ac-Lys)Lys-, Ac-Lys-(Lys)Lys-, Lys-(Lys)LysLys-(Ac-Lys)Lys-NH₂, Lys-(Lys) Lys-NH₂;
Ac-(Ac-Lys-Lys)-Lys-, (Ac-Lys-Lys)-Lys-, Ac-(Lys-Lys)-Lys- and (Lys-Lys)-Lys(Ac-Lys-Lys)-Lys-NH₂, and (Lys-Lys)-Lys-NH₂.

31. The exendin-4 peptide analogue according to the preceding claims, wherein the branched amino acid probe is selected from the group consisting of Ac-(Ac-Lys)Lys-, Ac-(Lys)Lys-, (Ac-Lys)Lys-NH₂, (Lys)Lys-NH₂ and (Lys)Lys-.

32. The exendin-4 peptide analogue according to the preceding claims, wherein (aa₃) is an amino acid residue selected from Gly and Ala.

33. The exendin-4 peptide analogue according to the preceding claims, wherein said first amino alkyl amino acid residue is covalently linked to the N-terminus of said exendin-4 peptide analogue.

34. The exendin-4 peptide analogue according to the preceding claims, wherein said first amino alkyl amino acid residue is covalently linked to the side chain amino group of a lysine or ornithine residue within said exendin-4 peptide analogue.

35. The exendin-4 peptide analogue according to the preceding claims, wherein said first amino alkyl amino acid residue is covalently linked to the ε-amino group of a lysine residue within said exendin-4 peptide analogue.

36. The exendin-4 peptide analogue according to the preceding claims, wherein said first amino alkyl amino acid residue is covalently linked to the side chain amino group of lysine at position 27 of said exendin-4 peptide analogue (Lys27).

37. The exendin-4 peptide analogue according to the preceding claims, wherein said first amino alkyl amino acid residue is covalently linked to the side chain amino group of lysine at position 12 of said exendin-4 peptide analogue (Lys12).

38. The exendin-4 peptide analogue according to the preceding claims, wherein said first amino alkyl amino acid residue is covalently linked to the C-terminus of said exendin-4 peptide analogue.

39. The exendin-4 peptide analogue according to the preceding claims comprising 1 branched amino acid probe.

40. The exendin-4 peptide analogue according to the preceding claims comprising 1 branched amino acid probe covalently linked to the N-terminus of said exendin-4 peptide analogue.

41. The exendin-4 peptide analogue according to the preceding claims comprising 1 branched amino acid probe covalently linked to the C-terminus of said exendin-4 peptide analogue.

42. The exendin-4 peptide analogue according to the preceding claims comprising 1 branched amino acid probe covalently linked to the side chain amino group of an amino alkyl amino acid residue within said exendin-4 peptide analogue; such as covalently linked to the δ-amino group of an ornithine residue within said exendin-4 peptide analogue or the ε-amino group of a lysine residue within said exendin-4 peptide analogue.

43. The exendin-4 peptide analogue according to the preceding claims comprising 2 branched amino acid probes.

44. The exendin-4 peptide analogue according to the preceding claims comprising 2 branched amino acid probes, wherein
i) one branched amino acid probe is covalently bound to the N-terminus of the exendin-4 peptide and another branched amino acid probe is covalently bound to the C-terminus of the exendin-4 peptide; or
ii) one branched amino acid probe is covalently bound to the N-terminus of the exendin-4 peptide and another branched amino acid probe is covalently linked to the side chain amino group of an amino alkyl amino acid residue within said exendin-4 peptide analogue; or
iii) one branched amino acid probe is covalently bound to the C-terminus of the exendin-4 peptide and another branched amino acid probe is covalently linked to the side chain amino group of an amino alkyl amino acid residue within said exendin-4 peptide analogue; or
iv) each of the two branched amino acid probes are covalently linked to the side chain amino group of separate amino alkyl amino acid residues within said exendin-4 peptide analogue.

45. The exendin-4 peptide analogue according to the preceding claims comprising 3 branched amino acid probes.

46. The exendin-4 peptide analogue according to the preceding claims comprising 3 branched amino acid probes, wherein
i) the first branched amino acid probe is covalently bound to the N-terminus of the exendin-4 peptide, the second branched amino acid probe is covalently bound to the C-terminus of the exendin-4 peptide and the third branched amino acid probe is covalently linked to the side chain amino group of an amino alkyl amino acid residue within said exendin-4 peptide analogue; or
ii) the first branched amino acid probe is covalently bound to the N-terminus of the exendin-4 peptide, and the second and third branched amino acid probes are each covalently linked to the side chain amino group of different amino alkyl amino acid residues within said exendin-4 peptide analogue; or
iii) the first branched amino acid probe is covalently bound to the C-terminus of the exendin-4 peptide, and the second and third branched amino acid probes are each covalently linked to the side chain amino group of different amino alkyl amino acid residues within said exendin-4 peptide analogue; or
iv) each of the first, the second and the third branched amino acid probes are covalently linked to the side chain amino group of different amino alkyl amino acid residues within said exendin-4 peptide analogue.

47. The exendin-4 peptide analogue according to the preceding claims, wherein said exendin-4 peptide is His-Gly-Glu-Gly-Thr-Phe-Thr-Ser-Asp-Leu-Ser-LysGln-Met-Glu-Glu-Glu-Ala-Val-Arg-Leu-Phe-Ile-Glu-Trp-Leu-Lys-Asn-Gly-GlyPro-Ser-Ser-Gly-Ala-Pro-Pro-X₁ (SEQ ID NO:4), wherein X₁ is selected from the group consisting of Ser and Pro,), or a functional variant thereof.

48. The exendin-4 peptide analogue according to the preceding claims, wherein said exendin-4 peptide is des-Pro³⁸-exendin-4(1-39) (SEQ ID NO:1)), or a functional variant thereof.

49. The exendin-4 peptide analogue according to the preceding claims, wherein said exendin-4 peptide is des-Ser³⁹-exendin-4(1-39) (SEQ ID NO:2)), or a functional variant thereof.

50. The exendin-4 peptide analogue according to the preceding claims, wherein said exendin-4 peptide is exendin-4(1-39) (SEQ ID NO:3), or a functional variant thereof.

51. The exendin-4 peptide analogue according to the preceding claims, wherein said exendin-4 peptide is a functional variant having one or more amino acid substitutions, such as one or more conservative amino acid substitutions.

52. The exendin-4 peptide analogue according to the preceding claims, wherein said exendin-4 peptide is C-terminally amidated (—NH₂).

53. The exendin-4 peptide analogue according to the preceding claims, wherein the C-terminus of the exendin-4 peptide is the unmodified C-terminal carboxylic group.

54. The exendin-4 peptide analogue according to the preceding claims, wherein said exendin-4 peptide is N-terminally acetylated (COCH₃ or Ac-).

55. The exendin-4 peptide analogue according to the preceding claims, wherein the N-terminal His of the exendin-4 peptide is the free amino moiety (H-His).

56. The exendin-4 peptide analogue according to the preceding claims, wherein said exendin-4 peptide, or a functional variant thereof;
i) retains the function of the non-variant or original sequence, at least to some extent,
ii) binds to and activates GLP-1R,
iii) is an agonist of GLP-1R,
iv) has binding affinity and/or receptor efficacy for GLP-1R.
v) stimulates glucose-dependent insulin secretion by pancreatic beta-bells,
vi) lowers blood glucose in mammal, and/or
vii) slows down gastric emptying.

57. The exendin-4 peptide analogue according to the preceding claims, wherein said exendin-4 peptide analogue is SEQ ID NO:1—(Ac-Lys-Lys)Lys-NH₂ (His-Gly-Glu-Gly-Thr-Phe-Thr-Ser-Asp-Leu-Ser-Lys-Gln-Met-Glu-Glu-Glu-AlaVal-Arg-Leu-Phe-Ile-Glu-Trp-Leu-Lys-Asn-Gly-Gly-Pro-Ser-Ser-Gly-Ala-ProPro-Ser-(Ac-Lys-Lys)Lys-NH₂).

58. A pharmaceutical composition comprising an exendin-4 peptide analogue according to any of the preceding claims.

59. An exendin-4 peptide analogue according to any of the preceding claims for use as a medicament.

60. An exendin-4 peptide analogue according to any of the preceding claims for use in the treatment of diabetes mellitus type 2.

61. An exendin-4 peptide analogue according to any of the preceding claims for use in the treatment of obesity.

62. An exendin-4 peptide analogue according to any of the preceding claims for use in enhancing satiety and/or reducing appetite.

63. An exendin-4 peptide analogue according to any of the preceding claims for use in a method of one or more of
a. glycemic control,
b. lowering blood glucose,
c. stimulating glucose-dependent insulin secretion by pancreatic beta-bells,
d. increasing insulin secretion in response to eating, and/or
e. suppressing release of glucagon in response to eating.

64. An exendin-4 peptide analogue according to any of the preceding claims for use in the treatment of an ischemic condition, an inflammatory condition, an infection and/or a metabolic condition.

65. The exendin-4 peptide analogue according to any of the preceding claims, wherein said exendin-4 analogue is administered simultaneously, sequentially or separately in combination with one or more further active ingredients.

66. The exendin-4 peptide analogue according to any of the preceding claims, wherein said exendin-4 analogue is administered simultaneously, sequentially or separately in combination with one or more further active ingredients selected from an oral glucose-lowering compound and insulin.

67. A method for treatment of diabetes mellitus type 2, obesity and/or enhancing satiety, said method comprising administering an exendin-4 peptide analogue according to any of the preceding claims to an individual in need thereof.
